(19) United States
(12) Patent Application Publication
(10) Pub. No.: US 2024/0423108 A1
(43) Pub. Date: Dec. 26, 2024

(54) SYSTEMS DEVICES AND METHODS FOR AGRICULTURAL WEEDING

(71) Applicant: AgriPass Robotics LTD., Givataim (IL)

(72) Inventors: Guy RAZ, Binyamina (IL); Yoav KOSTER, Kadima Zoran (IL); Assaf FRIDLER, Ramar Efal (IL)

(21) Appl. No.: 18/753,256

(22) Filed: Jun. 25, 2024

Related U.S. Application Data
(60) Provisional application No. 63/523,185, filed on Jun. 26, 2023.

(51) Int. Cl. A01B 39/18; A01B 79/00

(52) U.S. Cl. CPC A01B 39/18; A01B 79/005; G01N 33/245

(57) ABSTRACT

There are provided methods and systems for adaptively and selectively tilling soil for agricultural weeding comprising a sensing module configured and enabled to capture sensory data of the soil a mechanical module comprising at least one implement configured and enabled to perform tilling in the soil, a control module comprising: a communication circuitry and one or more processors, wherein the one or more processors are configured and enabled to process and analyze the captured sensory data to generate agricultural data of the soil; analyze the agricultural data and additional data to yield weeding strategy instruction signals; and transmit the weeding strategy instructions signals to the mechanical module for adaptively and selectively till or weed the soil.

### CROSS-REFERENCE

The present application claims priority to U.S. Provisional Application Ser. No. 63/523,185, filed on Jun. 26, 2023 entitled “ADVANCED TILLING SYSTEMS DEVICES AND METHODS FOR AGRICULTURAL WEEDING”, which is incorporated herein by reference in its entirety.

### TECHNICAL FIELD

The present invention, in some embodiments thereof, relates to a localized tilling systems devices and methods, and more specifically, but not exclusively, to tilling systems devices and methods for agricultural weeding using sensing devices and methods.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND OF THE INVENTION

Since its dawn some ten thousand years ago, agriculture was about battling weeds as much as it had been about cultivating the desired crops. Weeds pose a major issue and are one of the main factors that strongly affect yield via competition for nutrients, water, and sunlight. Untreated weeds can significantly diminish the yield of a given plot even to a state of minimal to no yield at all.

With the introduction of chemical herbicides, growers were able to battle weeds effectively improving yield by tens of percent. However, there is a growing need for agricultural weeding that would not rely on use of herbicides. This need stems from three main factors:
1. With the strong selective pressure herbicides introduced into the ecosystems, weeds are quick to develop herbicide resistivity, significantly reducing herbicides' effectiveness.
2. Herbicides are negatively affecting human health and the environment.
3. Herbicides economical cost is large.

Prior to the use of herbicides, and to this day, growers have used tilling to address weed problems. Tilling involves cultivating the top layer of the ground by a moving, typically towed, appropriate tool that cuts through the ground. This action can successfully remove emerged weeds. However, there are few deficiencies that might be associated with such tilling:
1. Tilling accelerates ground erosion and might lead to a depletion of essential ground nutrients. Tilling also leads to increased water evaporation and surface water runoff increasing soil water losses, floods and poor water infiltration into the soil.
2. Tilling increases the amount of Green House Gasses (GHG) like CO2 emitted to the atmosphere due to the agriculture process.
3. Tilling affects the soil's biological ecosystem harming benefactory factors that assist crop growth.
4. By turning the ground, tilling helps exposing new buried seeds (from the always existing seed bank) to appropriate conditions and actually encourages additional weeds growth.
5. Tilling requires large energy spent for large area cultivation.
6. Working in areas where the desired crop exists is tricky as the desired crop should not be harmed by tilling.

There is therefore a need for weeding systems, devices and methods that can effectively remove weeds without the negative impacts associated with tilling or the use of herbicides. Such a system will allow transitioning to sustainable agriculture practices, promoting healthy soil needed to support the growing demand for food supply.

### SUMMARY OF THE INVENTION

In accordance with a first embodiment of the present invention, there is provided a system for adaptively and selectively tilling soil for agricultural weeding, the system comprising: a sensing module comprising at least one sensor configured and enabled to capture sensory data of the soil; a mechanical module comprising at least one implement, wherein said at least one implement is configured and enabled to perform tilling in the soil; a control module comprising: a communication circuitry for communicating with said sensing module and said mechanical module; and a processing module, wherein said processing module comprising: one or more processors, wherein said one or more processors are configured and enabled to: process and analyze the captured sensory data to generate agricultural data of the soil; analyze the agricultural data and additional data to yield weeding strategy instruction signals; and transmit the weeding strategy instructions signals to the mechanical module for adaptively and selectively till or weed the soil.

In an embodiment, the processing module comprises: a detection module configured and enabled to analyze the sensory data to mark and discriminate plants from non-plants in said soil; a classification module configured and enabled to analyze the sensory data to distinguish different plant types in said soil; and a localization module configured and enabled to analyze the sensory data to identify the location of a plant's elements in the soil.

In an embodiment, the detection module or classification module are based on computer vision algorithms utilizing shape or color features.

In an embodiment, the detection module or classification module are based on one or more machine learning algorithms.

In an embodiment, the one or more machine learning algorithms are trained using labeled data.

In an embodiment, the one or more machine learning algorithms are based on deep learning algorithms, wherein said deep learning algorithms utilizing neural networks.

In an embodiment, the sensing module comprises at least one imager for capturing one or more images of the soil or a scene comprising the soil.

In an embodiment, the sensing module comprises an illumination module said illumination module comprises at least one illumination source.

In an embodiment, the sensing module is configured and enabled to construct a 2D or 3D model of the soil or scene.

In an embodiment, the at least one imager is selected from the group consisting of: an RGB camera, a monochrome camera, a thermal camera, a multi-spectral camera, a stereo camera, Time of Flight sensor, LIDAR sensor, RF sensor.

In an embodiment, the sensory data comprises one or more of: 2D or 3D images, and wherein a 2D or 3D model of the soil or scene are constructed based on said 2D or 3D images.

In an embodiment, the sensing module comprising at least two imagers, each imager having a predefined image capturing area and wherein there is a predefined overlap between the captured areas of the at least two imagers.

In an embodiment, the additional sensing module is configured and enabled to monitor the soil following the tilling action to provide quality assurance.

In an embodiment, the agricultural data comprises one or more of: crop or weeds type, growth stage, 2D or 3D location information of the crop or weeds, geometrical data, 3D structure of the scene. In an embodiment, the mechanical module comprises an end effector.

In an embodiment, the tilling action is conducted at a varying penetration depth.

In an embodiment, the at least one implement comprises: an upper section body for housing a motor said motor is configured and enabled to provide vertical motion of the implement with respect to the implement movement; at least one spring configured and enabled to lower the end effector into the soil based on the weeding strategy instructions signals.

In an embodiment, the motor is configured and enabled to rotate a strap for enabling the vertical movement of the implement's body along a first track.

The system of claim 18, wherein said at least one implement further comprises: a first spring connected to the end effector and to a second track, wherein said first spring is in a loaded state, and wherein the first spring is configured to vertically collapses for absorbing the impact along with the end effector to prevent it from breaking.

In an embodiment, the at least one implement further comprises: a second spring located at the bottom distal end of the implement and connected to the end effector, said second spring is configured to cause the end effector to fold upwards, parallel to the direction of the implement's movement.

In an embodiment, the mechanical module comprises: at least one row of implements, wherein said implements are arranged side by side, and wherein each implement of said implements covers a given width across the width of the mechanical module, and wherein each implement of said implements is configured and enabled moving up or down with respect to the movement of said mechanical module.

In an embodiment, the one or more processors are configured and enabled to: process the captured sensory data to extract a terrain profile of the soil.

In an embodiment, the mechanical module comprises at least two implements configured and enabled to follow the terrain profile of the soil to ensure optimal tilling action of the soil.

In an embodiment, the vertical motion of the at least one implement is split into two separate mechanisms a first mechanism capable of operating slow motion of up to 500 mm/sec and a second mechanism capable of fast motion in the range 800-1000 mm/sec.

In an embodiment, the slow motion of the at least one implement is configured and enabled to adjust the height of the at least one implement above the soil and to follow said extracted terrain following movement.

In an embodiment, the fast motion is configured to conduct a tilling action.

In an embodiment, the in the slow motion two or more implements are joined whereas in the fast motion each implements moves vertically separately.

In an embodiment, the mechanical module comprises a mechanism that allows forward motion compensation (FMC).

In an embodiment, the mechanical module comprises force limiters.

In an embodiment, the end effector comprises one or more of: a blade, a rod, a moving blade, a saw.

In an embodiment, the mechanical module comprises a force gauge.

In an embodiment, the additional data comprises one or more of: rules, vehicle's data, pre-configured data, 2D or 3D structure, local or external sensor's data

In an embodiment, the rules include one or more of: Match each weed type stage and location in said soil with appropriate tilling size and depth; Use location of crop in said soil to prevent tilling action that would endanger the crop; Obtain an optimal terrain following elevation of each implement above ground that would allow optimal tilling; Limit the simultaneous tilling action in order to prevent harm to the mechanical module or to optimize power consumption and efficiency; Prioritize weeding importance in case the limit above does not allow weeding of all the weeds; Monitor the location of the system and its forward motion in order to time correctly the tilling action of the at least one implement.

In an embodiment, the weeding strategy instructions signals comprise one or more of the following instructions: avoid removing too small weeds that cannot harm the crop; avoid removing too large perennial weeds; avoid removing weeds too close to crop; avoid rocks and other obstacles;

till at soil level or at a shallow depth for broadleaves weeds; till at a larger depth for grass-like weeds and for large weeds.

In an embodiment, the system comprises a storage unit for storing said sensory data or additional data.

In an embodiment, the mechanical module is towed or shoved by a vehicle, said vehicle is selected from the group consisting of: an autonomous vehicle, a tractor, a dedicated drivable vehicle, a tele-operated vehicle, controlled from a different location.

In accordance with a second aspect of the present invention, there is provided a method for adaptively and selectively tilling soil for agricultural weeding, the method comprising: obtaining sensory data from a sensory module wherein said sensory module comprises at least one sensor configured and enabled to capture the sensory data of the soil and wherein the sensory data comprises one or more of 2D or 3D images of the soil; processing and analyzing the sensory data, using a processing module, to generate agricultural data related to the soil; analyzing the agricultural data and additional data to yield weeding strategy instructions signals; transmitting the weeding strategy instructions signals to a mechanical module for adaptively and selectively till the soil.

In an embodiment the mechanical module is towed or shoved by a vehicle.

In an embodiment, the sensory data is further analyzed based on vehicle's data and local or external sensors.

### BRIEF DESCRIPTION OF THE DRAWINGS

A better understanding of the features and advantages of the present disclosure will be obtained by reference to the following detailed description that sets forth illustrative embodiments, in which the principles of embodiments of the present disclosure are utilized, and the accompanying drawings.

FIG. 1A and FIG. 1B are respective upper side views and side views of a system for adaptively and selectively tilling soil, in accordance with embodiments;

FIG. 1C shows a mechanical module configured of sensing and weeding an agricultural field of a width of six meters, towed by a vehicle, in accordance with embodiments;

FIG. 1D shows a mechanical module configured of sensing and weeding an agricultural field of a width of two meters, towed by a vehicle, in accordance with embodiments;

FIG. 1E shows a view of the mechanical module of FIG. 1D, in accordance with embodiments;

FIG. 2A shows a schematic block diagram of a weeding system, configured and enabled to adaptively and selectively till soil, in accordance with embodiments;

FIG. 2B and FIG. 2C show an imagers configuration, in accordance with embodiment;

FIG. 3A shows a schematic illustration of the mechanical module, in accordance with embodiments;

FIG. 3B shows a top-side three-dimensional illustration of the weeding module, in accordance with embodiments;

FIG. 3C shows a slightly tilted top-side three-dimensional illustration of the weeding module, in accordance with embodiments;

FIG. 3D, FIG. 3E, FIG. 3F and FIG. 3G show various configurations of implements of the mechanical module, in accordance with embodiments;

FIG. 3H and FIG. 3I show respectively a cross-section view and a perspective side view of an implement, in accordance with embodiments;

FIG. 3J, FIG. 3K, FIG. 3L and FIG. 3M show examples of various types of end effectors shapes that may be connected or embedded to the edge of the implement, in accordance with embodiments;

FIG. 3N and FIG. 3O show, respectively, a perspective view and an image of the mechanical module, in accordance with embodiments;

FIG. 4A shows a schematic detailed block diagram of the control module, in accordance with embodiments;

FIG. 4B and FIG. 4C show an illustration of a terrain follow of the mechanical module, in accordance with embodiments;

FIG. 5 shows a flowchart of the weeding/tilling process, in accordance with embodiments;

FIG. 6A illustrates a flowchart of a method for advanced adaptive and selective agricultural weeding and/or tilling, using a single imaging device, in accordance with embodiments;

FIG. 6B illustrates a flowchart of a method for advanced adaptive and selective agricultural weeding and/or tilling, using a stereoscopic imaging device, in accordance with embodiments; and

FIG. 7A, FIG. 7B, FIG. 7C, FIG. 7D, FIG. 7E, FIG. 7F show examples of images captured by the sensing module and analyzed by the processing module in accordance with embodiments.

### DETAILED DESCRIPTION OF THE INVENTION

In the following description, various aspects of the invention will be described. For the purposes of explanation, specific details are set forth in order to provide a thorough understanding of the invention. It will be apparent to one skilled in the art that there are other embodiments of the invention that differ in details without affecting the essential nature thereof. Therefore, the invention is not limited by that which is illustrated in the figure and described in the specification, but only as indicated in the accompanying claims, with the proper scope determined only by the broadest interpretation of said claims.

The configurations disclosed herein can be combined in one or more of many ways to provide advanced adaptive and selective agricultural weeding systems devices and methods. Specifically, the systems and methods in accordance with embodiments include conducting agricultural tilling and/or weeding of the soil in a highly localized manner according to the existence of weeds and/or the weeds characteristics in order to create a local treatment of weeds without disturbing the rest of the soil surface and ecosystem where weeds do not currently exist. The systems devices and methods, in accordance with embodiments are configured and enabled to mimic the action of a human laborer using one or more sensors which are in communication automatic tools, at a rate of hundreds or thousands of weeds per second.

The system and methods in accordance with embodiments provide a ‘see and weed’ solution using the one or more sensors acting as the system's ‘eyes’ and advanced agricultural mechanical tools, communicating with the one or more implements for advanced adaptive and selective agricultural weeding/tilling.

Advantageously, the tilling/weeding, in accordance with embodiments, is selective since it only occurs in a limited and and/or specifically small demarcated zone surrounding a detected weed and/or defined area surrounding a detected appropriate weed. Whereas prior art solutions include using massive machines such as cultivators that use for example rotary or linear motion causing detrimental effects on soil integrity, due to their indiscriminate nature and tendency to disrupt the soil structure.

Furthermore, the tilling, in accordance with embodiments, is adaptive since its depth and length around the weed can be adjusted, for example continuously and in real-time, based on sensing and identifying the weed class and/or type and/or growth stage using an advanced mechanical implement that can adaptively cultivate the soil and remove weeds.

Additionally, in certain scenarios, penetrating the soil and performing underground tilling action may prove unnecessary, particularly when weeds can be effectively removed by above ground manipulation addressing the leaves. In such cases, embodiments of the present invention will recognize such cases and adapt accordingly. Advantageously the adaptive capability of the systems and methods of the present invention, eliminates the need for excessive soil disturbance, thereby enhancing the efficiency and sustainability of weed control practices.

In an embodiment, the systems, devices and methods in accordance with embodiments are also configured to determine the right mechanical implement for proficiently addressing the weed and utilize the suitable specific implement.

According to one embodiment, there is provided, a system for adaptively and selectively agricultural weeding comprising a sensing module comprising at least one sensor configured and enabled to capture sensory data of the soil and/or weeds and/or crop and a control module comprising: a computer storage unit for storing said sensory data, a communication circuitry for communicating with the sensing module and a processing module comprising one or more processors configured and enabled to: process the captured sensory data and generate agricultural data of the soil and/or weeds and/or crop; and analyze the agricultural data and additional data to yield weeding strategy instructions; and send the weeding strategy instructions to a mechanical module for adaptively and selectively till and/or weed the soil.

One or more components of the configurations disclosed herein can be combined with each other in many ways.

Prior to the detailed description of the invention being set forth, it may be helpful to set forth definitions of certain terms that will be used hereinafter.

As used herein and throughout the claims, the term “tilling” or “till” encompasses any penetration into the soil with some mechanical implement that can move some amount of the soil including any plant part that exists in the soil.

As used herein and throughout the claims, the term “weed” or “weeding” encompasses any plant in a field or soil other than the desired crop. The term “weed” or “weeding” comprises any type of annual, bi-annual, perennial or any other growth plant. Additionally, “weed” or “weeding” may apply to residual previous years' crops growing under crop rotation or change of crop scheme, or any crop resulting from neighboring or other plots. For instance, soybeans can be classified as a “crop” during an initial year of cultivation, such as in the first year of an agricultural cycle, whereas in subsequent years or any chosen year, they may be considered as a “weed”.

As used herein, like characters refer to like elements.

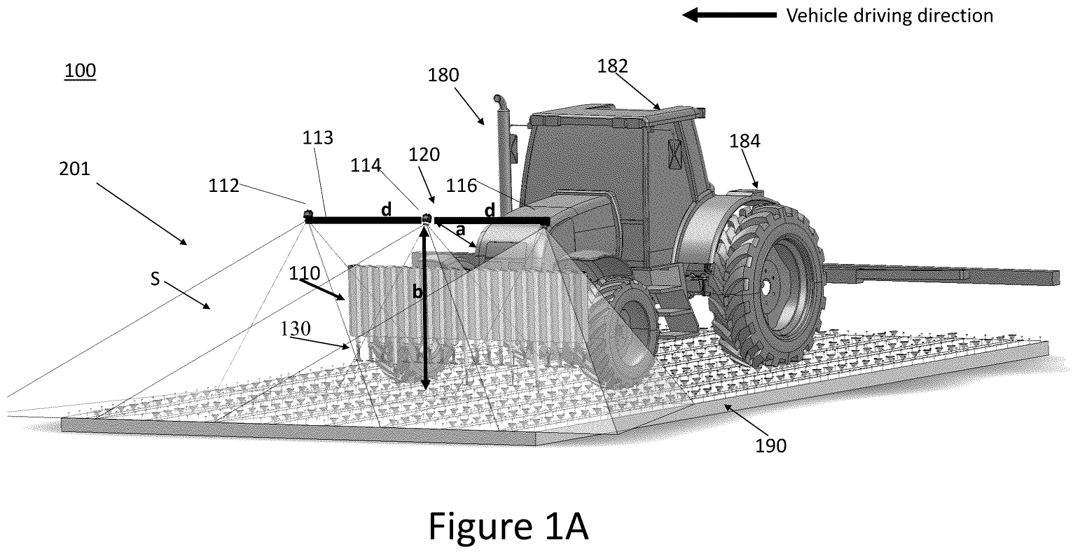

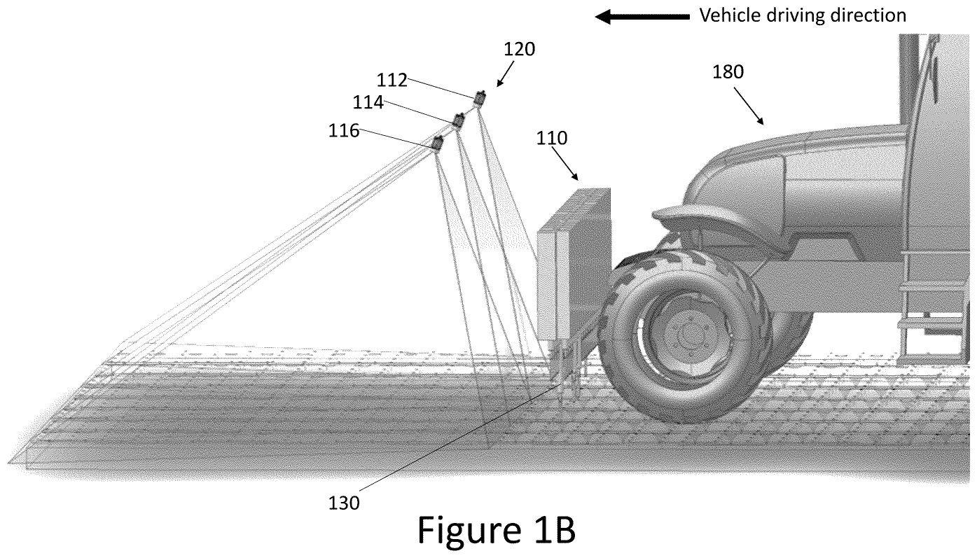

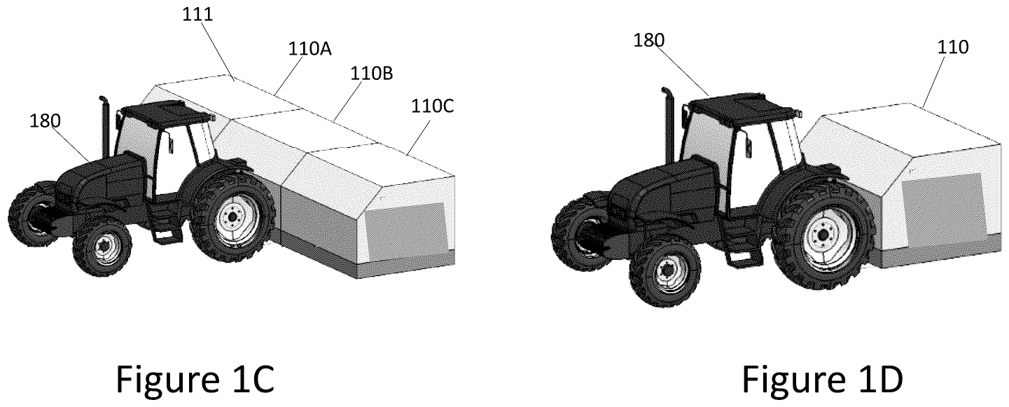

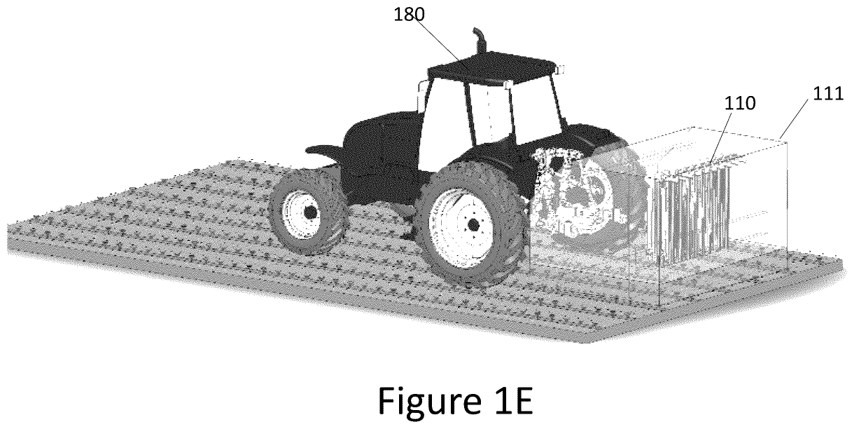

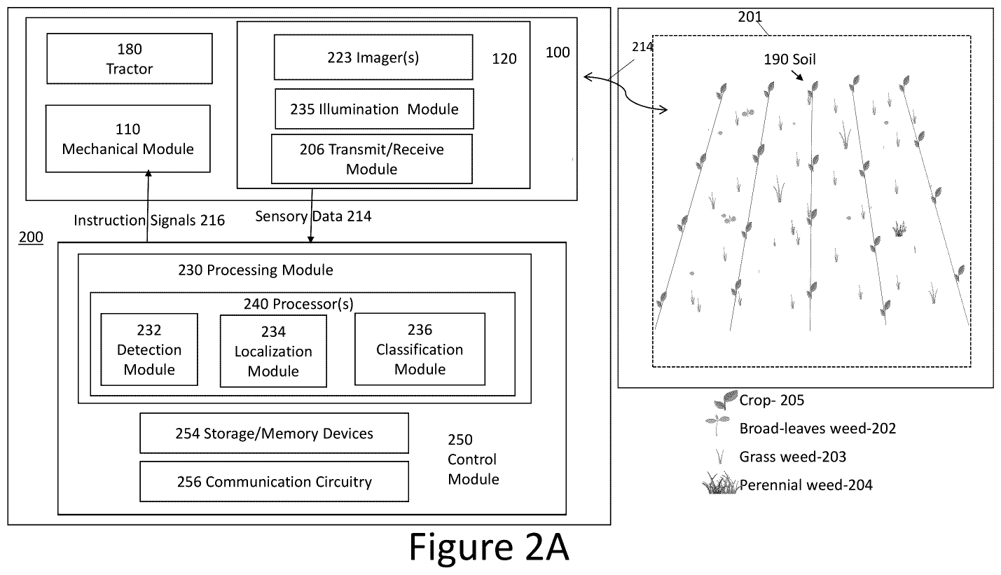

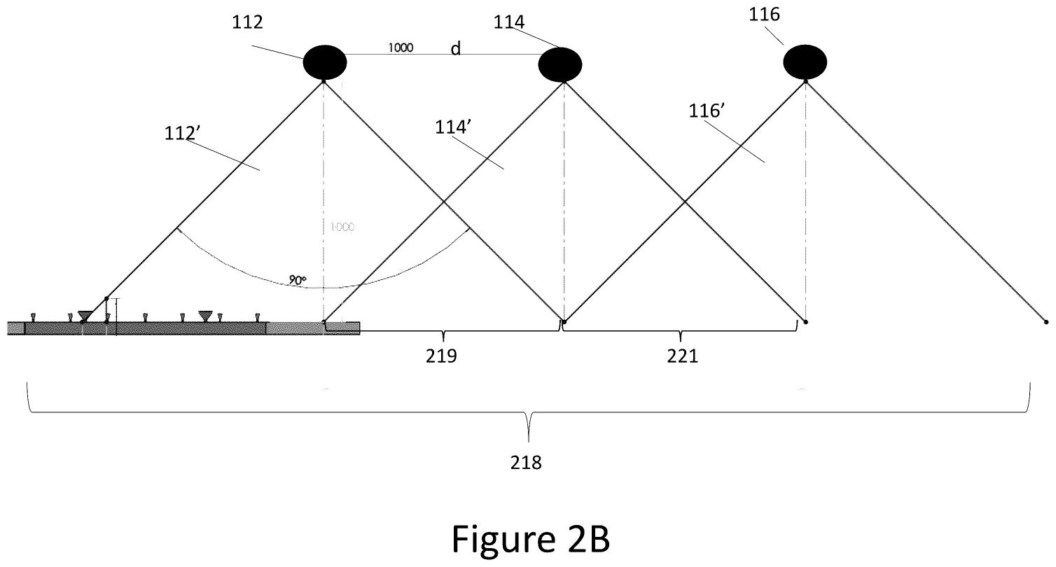

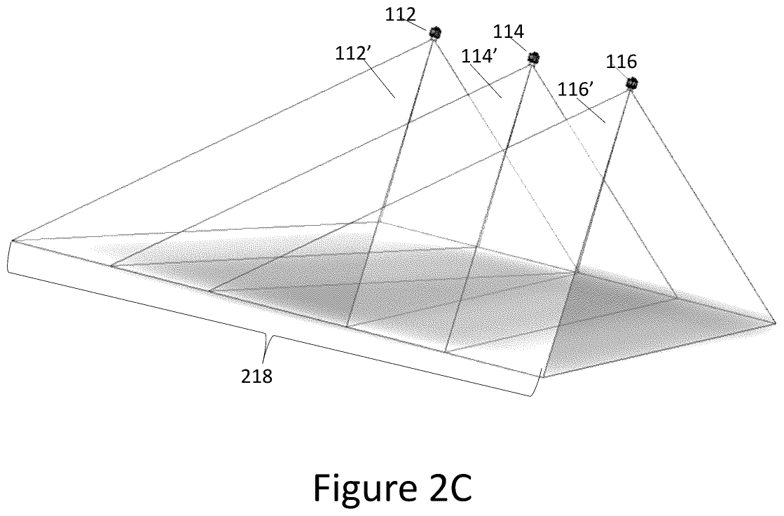

Referring now to the drawings, FIG. 1A and FIG. 1B show, respectively, perspective upper left side view and perspective left side view of a system 100 for adaptively and selectively tilling soil 190, in accordance with embodiments. System 100 comprises a mechanical module 110 which, for example, may be mounted, on one or more vehicles such as tractor 180, a sensing module 120, comprising one or more sensors such as three sensors 112, 114, and 116, and a processing module 230 (illustrated in FIG. 2A) comprising one or more processors which may be in communication with the sensing module 120 and/or the mechanical module 110.

According to one embodiment, the sensing module 120 is configured and enabled to sense a scene 201 (e.g. sensed area) including soil and/or soil planted area 190 which needs to be handled for weeding and generating sensory data 214 corresponding to the scene 201 and specifically to soil 190 in the sensed area.

In some cases, the sensed area may be for example in a size of between 0.5 to 20 meters wide and 0.5 to 2 meters long. The sensing module 120 (sensors 112, 114 and 116) may be attached to a pole, such as a rigid baseline 113 connected to the vehicle 180, for example outside the vehicle cabin in the front and/or back section of the vehicle for example, in proximity to the mechanical module 110. Specifically, as shown in FIG. 1A three sensors 112, 114 and 116 may positioned in the front section of the tractor 180. In a preferred embodiment, each of the three sensors 112, 114 and 116 may be a stereo camera sensor positioned for example at a distance ‘d’=1 m from each other, and at a distance ‘a’ from the vehicle and at a distance ‘b’ from the soil 190.

As an example, the stereo camera may include two RGB cameras, mounted at a small distance from each other, such as 15 cm apart on a rigid baseline.

Each of the sensors 112, 114 and 116 may have a respective Field of View (FoV, defined for example as a predefined image capturing area) 112′, 114′ and 116′ (shown in FIG. 2B).

These FoVs may be arranged in such a manner that their respective coverage areas overlap or intersect with one another. By positioning and configuring the FoVs to have overlapping regions, system 100 ensures an optimal and comprehensive view of the target area or surface of the scene 201 (and soil 190). The overlapping FoVs provide several advantages, including:
Redundancy and fault tolerance: If one FoV experiences a malfunction or obstruction, the overlapping regions from the other FoVs can compensate for the missing or corrupted data, ensuring continuous and reliable monitoring of the soil 190 and/or scene 201.
Enhanced coverage: The overlapping FoVs eliminate potential blind spots or gaps in the field of view, resulting in complete and seamless coverage of soil 190 and/or scene 201.
Multi-perspective analysis: With multiple FoVs capturing the same region from different angles or perspectives, the system 100 can perform more comprehensive analysis, such as stereoscopic imaging, depth perception, or triangulation, enabling advanced sensing and measurement capabilities.
Increased resolution: By combining and fusing the data from overlapping FoVs, the system 100 can effectively increase the effective resolution or detail level of the captured information, leading to improved analysis and decision-making.
   The one or more sensors 112, 114, and 116 may be one or more of the following sensors, or a combination thereof: Camera; Multi-spectral camera; Thermal camera; Stereo-camera; Time of Flight (TOF) sensor; Light Detection and Ranging (LIDAR) sensor; Radio Frequency (RF) sensor such as a Radar sensor; Ultrasonic (US) sensors.

According to one embodiment, the mechanical module 110 is an agricultural weeding/tilling system configured and enabled to selectively provide a highly localized footprint using a plurality of implements 130 such as anchors configured and enabled to accurately penetrate the ground (e.g. soil 190) and selectively till preselected locations in the soil 190. In accordance with embodiments, the implements 130 are one or more actuators such as mechanical actuators for enabling control and precise control of movement or force for tilling the soil. In some embodiments, the selectively provided highly localized footprint may be in the range of 1-5 cm×1-5 cm soil disturbance around each weed.

The sensory data 214 is transmitted to the processing module 230, where the received sensory data 214 is analyzed and processed, using one or more processors 230.

The one or more processors 230 are configured and enabled to receive the sensory data 214 and analyze the sensory data including detecting and/or classifying and/or localizing weeds and/or crop in the soil 190, and/or including mapping the 3D structure of the soil and plant, and accordingly provide instructions (e.g. Instruction Signals 216) to the mechanical module 110 to activate (e.g. selectively activate) the implements 130 to provide localized tilling based on the processed sensory data 214 which includes the detected and classified characteristics.

For example, the mechanical module 110 may be housed within an agricultural enclosure 111 and towed by the tractor 180, as illustrated in FIGS. 1B and 1C. This arrangement ensures the module is protected and can be easily transported and utilized in various agricultural tasks.

Alternatively or in combination, the vehicle may be an autonomous vehicle, or a weeding robot and the mechanical module 110 may be fitted on the autonomous vehicle.

Alternatively or in combination, the vehicle may be a dedicated drivable vehicle, and the mechanical module 110 may be part of the dedicated drivable vehicle.

Alternatively or in combination, the vehicle may be a tele-operated vehicle that is controlled remotely by an operator such as tele-operated vehicle controlled from different location and the mechanical module 110 may be mounted on the tele-operated vehicle.

According to one embodiment, the sensing module 120, the processing module 230 and the mechanical module 110 may be installed on a single device or system. In other embodiments, the system's 100 modules may be separated and located at various locations. For instance, the mechanical module could be mounted on the vehicle, like the tractor 180 while the software modules such as the processing and storage could be on the cloud. For example, the sensing module 120 may be shoved at the front of a tractor 180 and may be in communication, with the mechanical module 110 which is towed at the rear section of the tractor. It is stressed that such a separated configuration might provide additional packaging solutions but would require accurate co-registration between the two modules to ensure for example that the sensing module 120 and the mechanical module 110 are correctly communicated with respect to the crop and weed location.

FIG. 2A shows a schematic block diagram of a system such as a weeding system 200, configured and enabled to adaptively and selectively perform weeding and/or tilling operations on soil 190, in accordance with embodiments. In some cases, the weeding system 200 may be or may include system 100 of FIGS. 1A and 1B. System 200 comprises a vehicle (e.g. tractor 180), mechanical module 110, sensing module 120, and a control module 250, in accordance with embodiments.

In accordance embodiments, each of the system's 200 modules is capable of independent operation and can establish communication with one another. For instance, a control module 250 can establish communication with the sensing module 120 and/or the mechanical module 110.

Optionally the sensing module 120 and the control module 250 are integrated together in a single device. In some cases, the sensing module 120 and the control module 250 are integrated separately in different devices.

Optionally, the modules, such as the processing module 230 can be integrated into one or more cloud-based servers.

Alternatively or in combination, the control module 250 and/or processors 240 may partially analyze the sensory data 214 prior to transmission to a remote processing and control unit. In some cases, the remote processing and control unit can be coupled to the mechanical module 110 or to a hand-held device (e.g. a cell phone). In some cases, the remote processing and control module 250 can be a cloud-based system which can transmit analyzed data or results to a user.

In an embodiment, the mechanical module 110 includes a chassis used as the frame of the mechanical module 110, support wheels, encoders which may be connected or embedded into the vehicle's' wheels and are configured to measure the mechanical module 110 forward motion, a power device (e.g. battery) and a stabilization device (e.g. suspension) comprising passive suspension system (between wheels and body) and/or active motorized stabilization.

In an embodiment there is a GPS receiver and/or GPS-RTK to measure the mechanical module location.

In accordance with one embodiment, the sensing module 120 comprises, for example, one or more imagers 223 (e.g. stereo camera) for capturing the sensory data 214 including, for example, images (e.g. 2D or 3D images) of scene 201 (e.g. soil 190) and specifically including imaging for example grass weed 203, Broad-leaves weed-202, perennial weed-204 and crop 205 in the scene, a transmit/receive module 206 for transmitting the captured sensory data 214 to the control module 250.

The control module 250 comprises a processing module 230 including one or more processor(s) 240, Storage and/or Memory devices 254 and communication circuitry 256.

Components of the control module 250 can be configured to transmit/receive, store, and/or analyze the captured sensory data 214 and generate instructions (e.g. instruction signals 216) transmitted to the mechanical module 110.

In some aspects, the control module 250, such as the one or more processors 240 in the processing module 230 may be implemented in software (e.g., subroutines and code).

In some cases, the processors 240 may comprise or may be a tangible medium embodying instructions, such as a computer-readable memory embodying instructions of a computer program. Alternatively or in combination the processors 230 may comprise logic such as gate array logic in order to perform one or more logic steps.

In some aspects, some or all of the modules may be implemented in hardware (e.g., an Application Specific Integrated Circuit (ASIC), a Field Programmable Gate Array (FPGA), a Programmable Logic Device (PLD), a controller, a state machine, gated logic, discrete hardware components, or any other suitable devices) and/or a combination of both. Additional features and functions of these modules according to various aspects of the subject technology are further described in the present disclosure.

The processing module 230 is responsible for performing the computations and data manipulation can be implemented in various configurations. In one embodiment, the processing module 230 is located locally, such as on the same device or system as the other components of the invention. For example, the processing module 230 could be integrated into a single chip, circuit board, or enclosed housing along with other necessary hardware elements. Alternatively, the processing module 230 could be a separate standalone unit that interfaces and communicates with the other local components over a wired or wireless connection. In another embodiment, the processing module 230 is located remotely from the other components, such as in a cloud computing environment or server accessed over a network like the internet. The remote processing module could be part of a centralized system that handles computations for multiple client devices or installations of the invention. The remote approach allows for offloading intensive processing tasks to high-performance cloud resources. Hybrid configurations are also possible, wherein certain processing tasks are divided between local and remote modules based on factors like computing demands, data access needs, and network capabilities.

According to some embodiments, the communication circuitry 256 comprises a data acquisition module configured and enabled to receive the sensory data 214 and perform one or more of: signal conditioning, analog-to-digital conversion, sampling, and data processing tasks to prepare the data for further analysis. Signal conditioning ensures data integrity by amplifying weak signals and filtering out noise, while analog-to-digital conversion facilitates compatibility with digital systems. Sampling captures sensor readings over time, and subsequent processing refines the data for analysis. Error detection and correction mechanisms ensure data integrity during transmission to computing systems or storage devices, facilitating various applications such as monitoring, control, and analysis.

In case the sensory data are RF signals, the communication circuitry 256 collects and digitizes the signals from the transmit/receive module 206 while tagging the signals according to the antenna combination used and the time at which the signals were collected. The communication circuitry 256 may include a data acquisition subsystem which typically includes analog-to-digital (A/D) converters and data buffers, but it may include additional functions such as signal averaging, correlation of waveforms with templates or converting signals between frequency and time domain.

Specifically, the one or more processors 240 are configured to analyze the captured sensory data 214 to extract visual data and/or depth data of scene 201 (e.g. soil 190) and generate the instruction signals 216 based on the analysis results. More specifically, the one or more processors 240 are configured and enabled to receive the sensory data 214 of the scene 201 and analyze the sensory data 214 to detect, classify and localize weeds of various types such as: Broad-leaves weed 202, Grass weed 203, Perennial weed 204 and crop 205 in the soil 190.

According to one embodiment, the processor(s) 240 comprises a detection module 232, a classification module 234 and a localization module 236.

The detection module 232 is configured and enabled to analyze the sensory data to mark and discriminate plants from non-plants in said soil. Specifically, the detection module 232 is configured and enabled to analyze the sensory data 214 and mark and discriminate plants from non-plants such as ground, rocks, strews, garbage, etc.

The classification module 234 is configured and enabled to analyze the sensory data to distinguish different plant types in said soil. Specifically, the classification module 234 is configured and enabled to analyze the sensory data 214 to identify and/or distinguish and/or classify different plant types. More specifically, the classification may address the following characteristics:
a. Classify weed vs. crop—e.g., weeds are to be handled, the crop is to be protected;
b. Class of weed—Broadleaf vs. grass as an example. Broadleaf plants might be handled by trimming all the leaves above ground level, leaving the roots intact. In contrast, grass weeds might regrow unless cut below the crown i.e. below ground;
c. Specific weed species—Identify specific species that may require adjusted treatment. As an example, specific perennial weeds may have a wide root system with tubers that may require deeper localized tilling.
d. Growth stage—Young vs. Older plant or age estimation-Younger weeds may not require handling as the crop may outpace them in growth and suppress them. On the other hand, older weeds, may require deeper tilling to uproot.
e. Weed size—Estimate the size of the weed to optimize the depth of the action in the soil to match the estimated size of the root system.

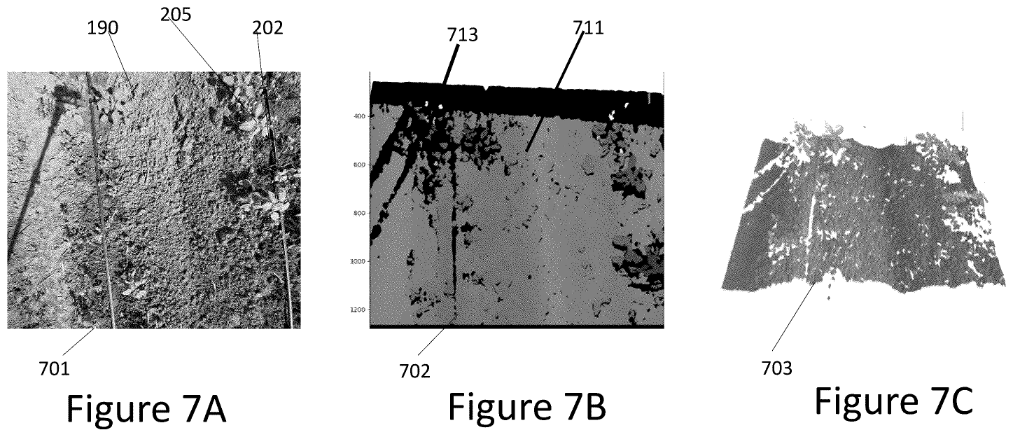

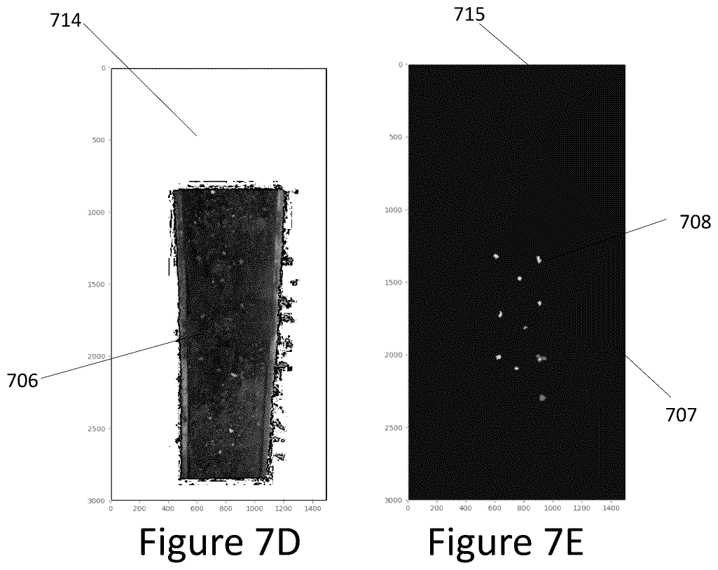

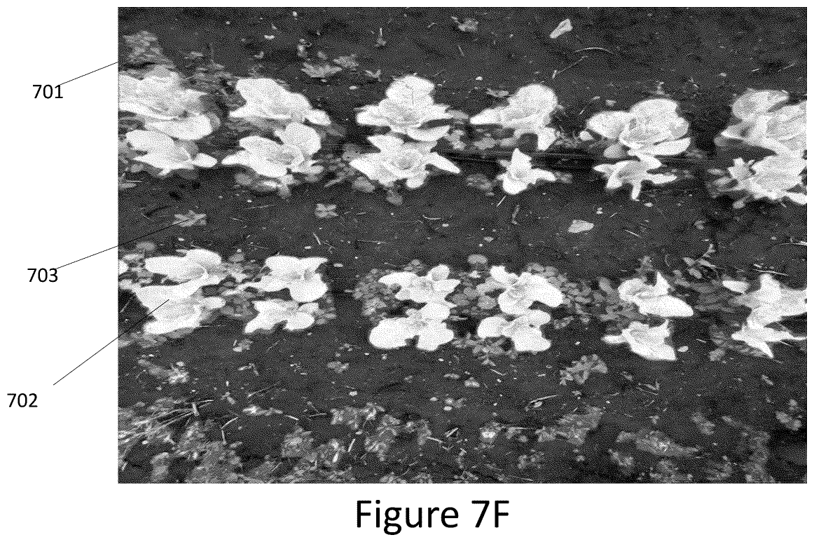

In accordance with embodiments the localization module 236 is configured and enabled to analyze the sensory data to identify the location of a plant's elements and/or weed in the soil as shown for example in FIG. 7E and FIG. 7F.

According to some embodiments, the detection and/or classification may be based on computer vision algorithms utilizing shape and color features. Alternatively or in addition, the detection and classification algorithms can utilize machine learning algorithms that are trained using labeled data. Specifically, the machine learning algorithms can be based on deep learning algorithms, utilizing neural networks, such as semantic segmentation and/or YOLO.

In the embodiment shown in FIG. 7F the classification module 236 is configured to do semantic segmentation in image 701 between Lettuce 702 (bright color mask) vs. weed 703 (dark color mask).

The localization module 236 is configured and enabled to analyze the sensory data 214 to identify the location of the plant's elements. For example, to generate a 2D or 3D location of the plant's elements with respect to the system's or camera's (e.g. mechanical module 110 and/or sensing module 120) coordinates framework. Specifically, the localization may include identifying the exact location of the stem origin in the ground, with respect to the sensing module 120 and the mechanical module 110 coordinate framework. In some cases, the exact location may be with an accuracy better than 5-10 mm.

In an embodiment, the relative location of the plants (e.g. crop 205) can further help in the classification process. As an example, the crop may be typically sowed in rows, making it likely that any plant that is out of the rows may be a weed. An algorithm that utilizes the localization to identify rows and hence classify everything that is out of the rows as weed can be utilized, in accordance with embodiments.

In accordance with embodiment, the localization includes transferring from the received image/sensors coordinates to 3D world coordinates with respect to the mechanical module coordinates system. In some cases, the transferring process may be based on geometry and assuming flat ground. Alternatively or in combination, a 3D model image of the scene 201 (e.g. soil 190) can be constructed in order to accurately localize the stem point of origin.

In operation, the Control Module 230 may send one or more Instruction Signals 216 to the towing or shoving platform of the Mechanical Module 110, be it the tractor 180 or other vehicle, in order to adjust the forward motion velocity according to the conditions detected by the sensing module 120. As an example, in case the sensing module 120 observes a high weed density in Soil 190 the Control module 250 may request the forward motion speed to be lowered. In contrast, if the observed weeds density is low, the control module 250 may send instruction signals allowing a faster motion. In some cases, the instruction signals can be also communicated to a human driver instructing the driver to manually adjust the tractor/weeding machine speed.

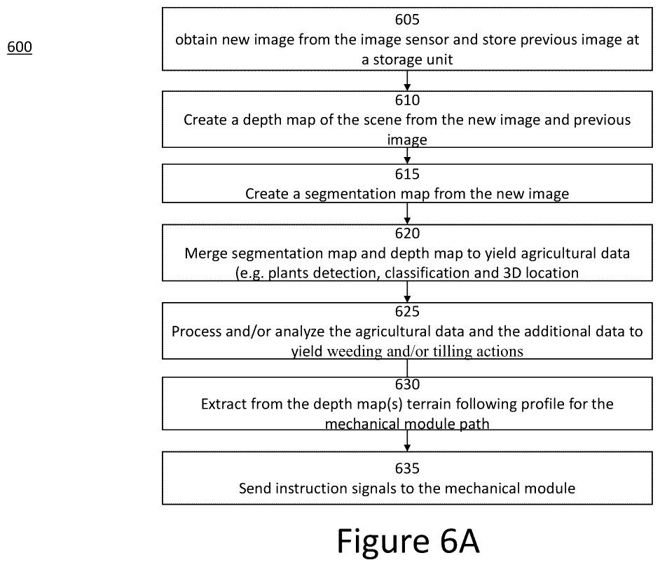

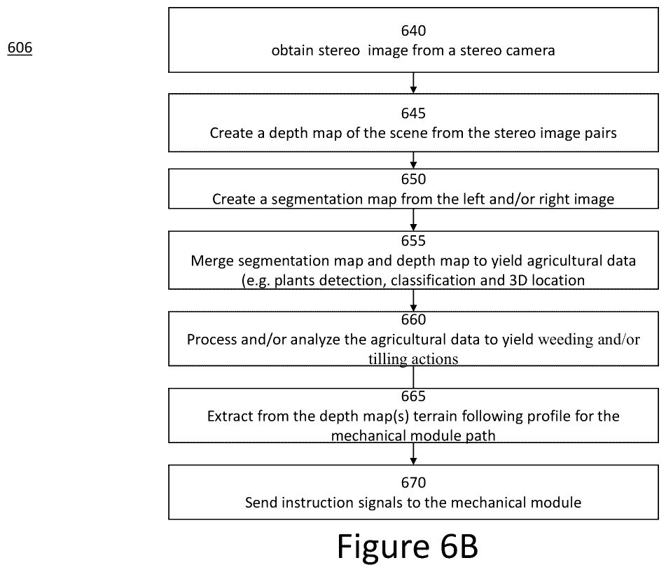

According to one embodiment, the sensing module 120 may be or may include a single camera, such as a 5 MP RGB camera. The single camera captures multiple images from varying viewpoints as the vehicle, such as the Tractor 180, moves through the soil 190. These varying viewpoints are used to construct a 2D model or three-dimensional (3D) model such as 3D model image of the scene 201 or soil 190. This technique, as illustrated in detail in FIG. 6A may utilize the principle of “structure from motion,” wherein the relative motion between the camera and the scene allows for the reconstruction of the 3D structure of the environment based on the captured two-dimensional (2D) images.

According to one embodiment, the sensing module 120 may include a ToF (Time-of-Flight) imaging device including one or more ToF sensors such as Continuous Wave Modulation (CWM) sensors or other types of ToF sensors for obtaining 3D data of the scene and/or one or more sensors for obtaining 2D of the scene.

According to one embodiment, the sensing module 120 may be a stereoscopic imaging device including one or more stereoscopic imagers for obtaining 3D data of the scene and one or more imagers for obtaining 2D of the scene. Specifically, the stereoscopic imagers may be of either visible light camera (e.g. preferred embodiment), multispectral camera, or thermal camera.

According to another embodiment, the imaging device may be or may include a LIDAR sensor.

According to one embodiment, the sensing module 120 may include a structured light imaging device including one or more imagers for obtaining 3D data of the scene and one or more imagers for obtaining 2D of the scene, as illustrated herein below in FIG. 1A and FIG. 1B.

In an embodiment, the sensing module 120 may include one or more visible cameras. The cameras can be monochrome or an RGB cameras, with the later having advantage to be able to use plants color for detection.

In an embodiment, the sensing module 120 may include one or more multi-spectral cameras. The multispectral cameras may include using different spectral channels to detect and identify plants in the scene. As an example, the measure of the Normalized Difference Vegetation Index (NDVI) defined as the difference between a NIR channel and a red channel, divided by their sum (NIR-R)/(NIR+R) can be used in order to identify vegetation material.

In an embodiment, the sensing module 120 may include one or more thermal imagers.

In an embodiment, the sensing module 120 comprises one or more Radio Frequency (RF) antenna sensors such as a Radar sensor and/or an antenna array.

In an embodiment, the sensing module 120 comprises one or more Ultra Sonic (US) sensors.

According to some embodiments, the 2D or 3D model images can be constructed, for example, based on the 2D or 3D images, using at least one of the above methods and devices.

Specifically, in an embodiment, sensing module 120 comprises an illumination module 235 comprises one or more illumination sources such as LEDs configured to illuminate the scene 201 (e.g. soil 190), and an imaging module, comprising one or more imagers 223, such as stereo camera(s) configured to capture 2D and/or 3D images of the scene 201. In some cases, the one or more imagers 223 may be cameras or video cameras of different types.

The illumination module 235 is configured to illuminate scene 201, using one or more illumination sources. In some embodiments, the illumination module 235 is configured to illuminate the scene 201 with broad-beamed light such as high-intensity flood light to allow good visibility of the scene 201 and accordingly for capturing images of the scene.

In an embodiment, the illumination module 235 is configured to operate in flash (strobe) mode and illuminate the scene 190 in sync and only during the exposure time of the imagers 223. Advantageously, such a method would lead to the illumination module 235 only operating in a low-duty cycle and hence reduce the amount of power and heat unnecessarily produced by the illumination.

In some embodiments, the illumination module 235 is configured to illuminate the scene 201 with structured light and accordingly capture 3D images of the scene. According to one embodiment, the structured light pattern may be constructed of a plurality of diffused light elements, for example, a dot, a line, a shape and/or a combination thereof. According to some embodiments, the one or more light sources, may be a laser and/or the like configured to emit coherent or incoherent light such that the structured light pattern is a coherent or incoherent structured light pattern.

According to some embodiments, the illumination module 235 is configured to illuminate selected parts of the scene 201.

According to some embodiments, imager 223 may be a CMOS or CCD sensor. For example, the imager(s) 223 may include a two-dimensional array of photo-sensitive or photo-responsive elements, for instance, a two-dimensional array of photodiodes or a two-dimensional array of charge coupled devices (CODs), wherein each pixel of the imager measures the time the light has taken to travel from the illumination module 235 (to the object and back to the focal plane array).

In some cases, the imagers 223 may further include one or more optical band-pass filter, for example for passing only the light with the same wavelength as the illumination sources.

In accordance with embodiments, the sensing module 120 is configured to generate sensory data 214 including for example visual images (e.g. 2D/3D images) and depth parameters of the scene, e.g., the distance of the detected objects to the sensing module 120. The sensory data 214 is analyzed and/or processed for example by the one or more processors 240 in the Processing module 230 to yield data including, for example, 3D data including the distance of the detected objects (e.g. weed and/or plants etc.) to the imagers 223 (e.g. depth maps) based on the obtained 3D data and the location in the image of the detected objects are combined in order to perform detection and/or classification and localization of elements in the soil 201 as will be described in further detail herein.

In some embodiments, the sensing module 120 may include a single imager or a plurality of imagers. For example, in one embodiment an array of imagers may be positioned in the back and/or front of a tractor 180 as shown in FIG. 1A and FIG. 1B, where, each imager may cover an imaging area having a size of, for example, a lateral width of 2-4 meters width and a longitudinal length of 1-3 meter or more.

In accordance with embodiments, the number of imagers included in the sensing module 120 may depend on the size of the related mechanical module 110 (e.g. weeding machine). For example, a wider machine of six meters width may then have three imagers spread such that the entire lateral width of six meters and a longitudinal length of 1 meter will be covered.

The mechanical module shown in FIG. 1C is configured of sensing and weeding an agricultural field of a width of six meters. In one embodiment, the system is modular, where each mechanical module 110 covering an area of for example two meters width. The user can connect multiple mechanical modules to accommodate the size of the field to be worked on. For example, the mechanical module may comprise three mechanical modules 110A, 110B and 110C, each covering a two meters width. The mechanical modules 110A, 110B and 110C may be connected one to the other according to the field's size. In case the agricultural field is small (e.g. 1, 2 meters or shorter in length), a respective smaller throughput may be required and hence the user may use a single mechanical module 110 as illustrated in FIG. 1D and FIG. 1E.

It should be noted that the specified ranges are exemplary, and the actual coverage may vary, including exceeding six meters.

In an embodiment, as illustrated in FIG. 2B there is an overlap between the respective Field of View 112′, 114′ and 116′ of each imager 112, 114, 116 such that each point of the regarded area 218 is seen from two different directions by two different imagers. For example, region 219 and region 221 are two overlapping regions, where region 219 is covered by both imagers 112 and 114 and region 221 is covered by both imagers 114 and 116. This configuration reduces the chances of weeds to be obscured from the sensing module due to the crop or due to other weeds.

In an embodiment, the mechanical module 110 is viewed within the field of view (e.g. field of view 112′, 114′ and 116′) of the sensing module 120.

FIG. 2C illustrates a respective upper left side view of a sensing module layout including the three imagers, in accordance with embodiments.

In another embodiment, the sensing module 120 may include a first and a second layer of imagers. For example, the first layer of imagers may include an array of imagers positioned in the front section of a weeding mechanism, for example imagers 112, 114 and 116, for imaging the soil prior to the weeding action and additional sensing module, such as a second layer of imagers may be fixed behind the weeding mechanism, for example on the roof 182 or above the back wheels 184 of the vehicle, in order to conduct a quality assurance process and provide feedback to the processing module as to the success of the weeding action. Such an assessment can help the user in assessing whether and when additional pass in the field may be needed and specifically at which locations. It may also provide guidance as to the existence of weeds that cannot be addressed by the weeding mechanism and hence require a different treatment such as manual weeding. The treatment can be for example repeating driving at a later time, sending manual workers, to complete the weeding etc.

In an embodiment, the processing involves the utilization of machine learning algorithms, and specifically deep learning algorithms in order to conduct the detection and/or the classification of the crop and weeds.

In another embodiment, the detection and the classification can be made utilizing different image characteristics. As an example, the detection of plants can be made using a full image that has been resized or binned to a lower resolution to reduce the amount of computation. As another example, the classification can be conducted only on small full-resolution cropped image parts that are cropped around the plant's detected image location. Such a method can considerably further reduce the amount of computations involved.

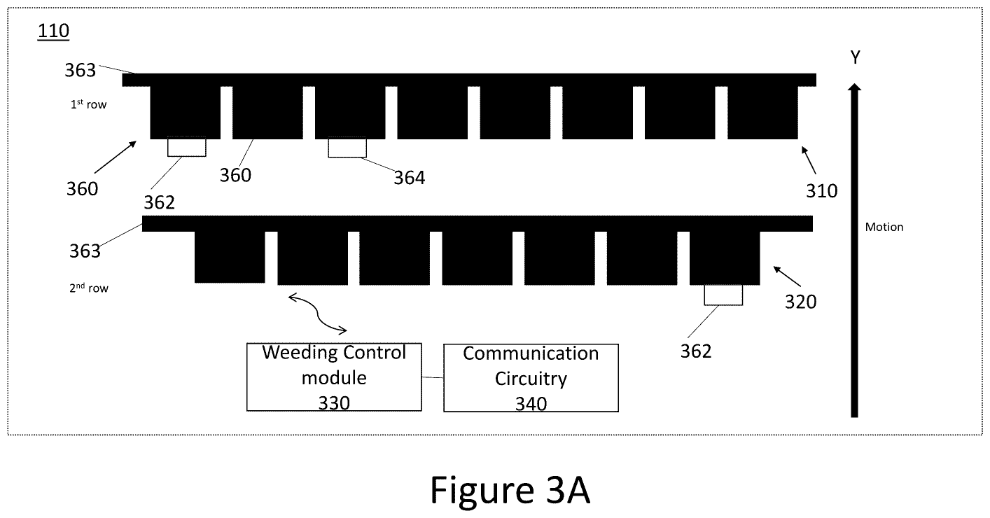

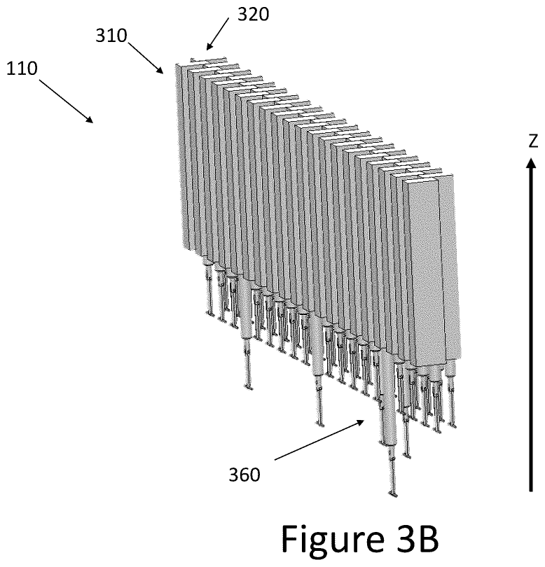

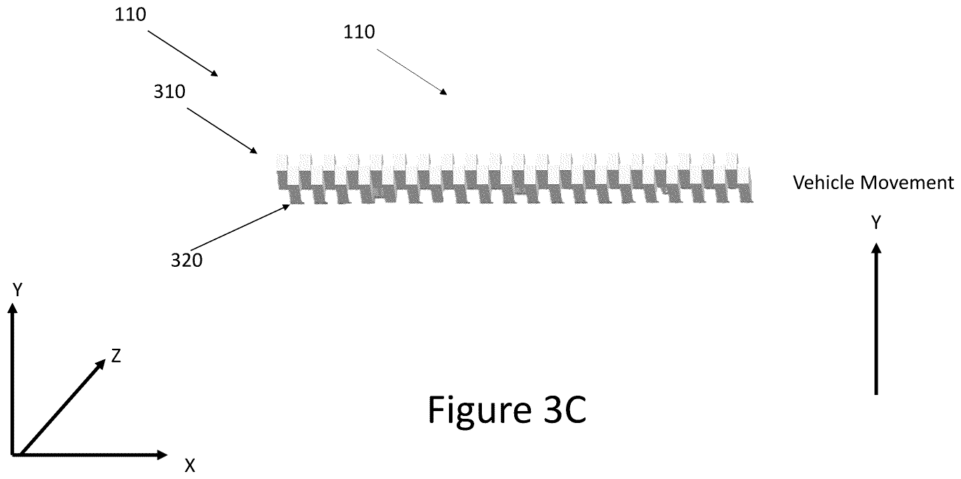

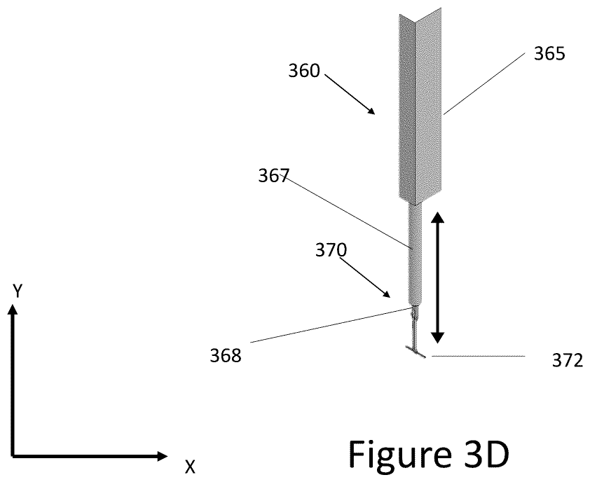

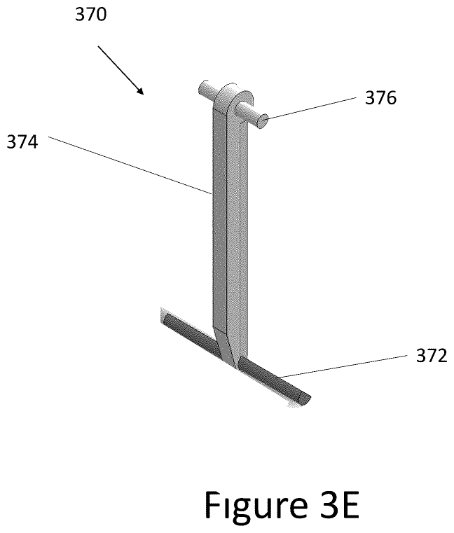

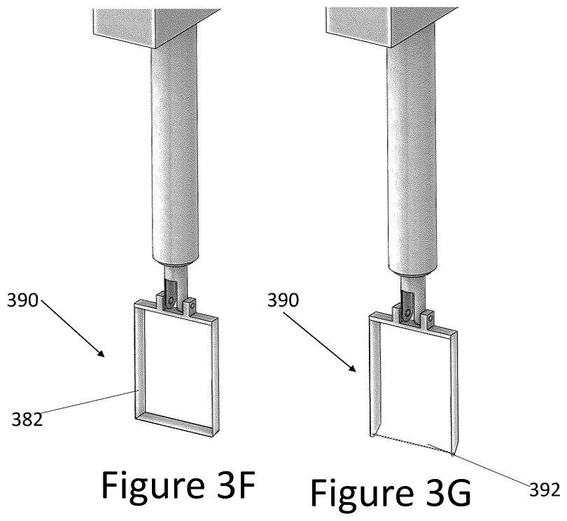

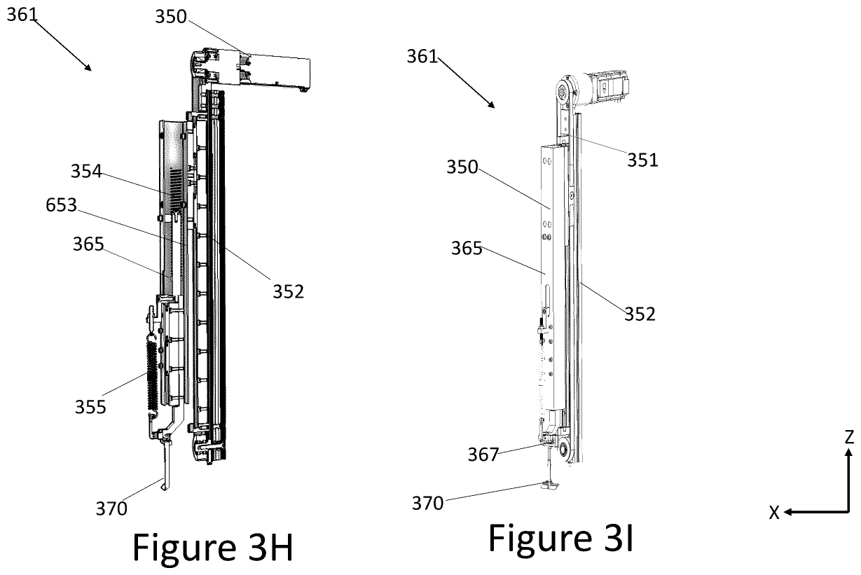

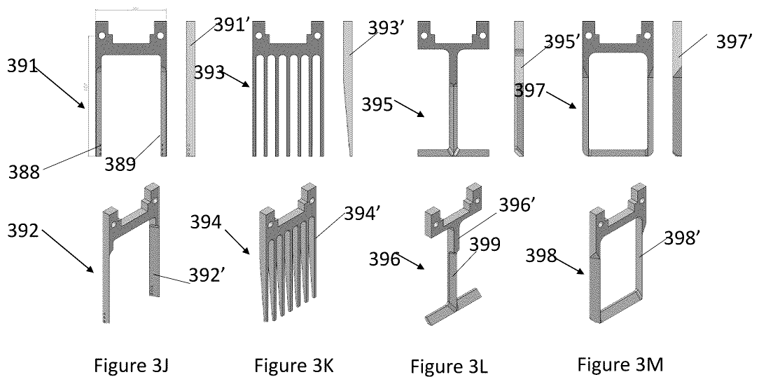

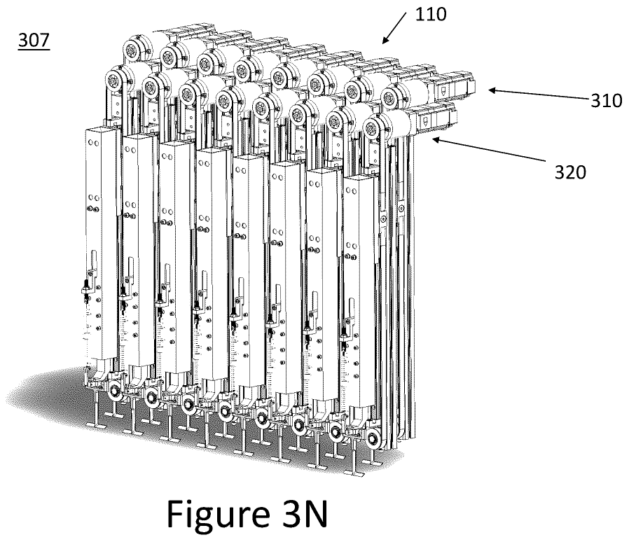

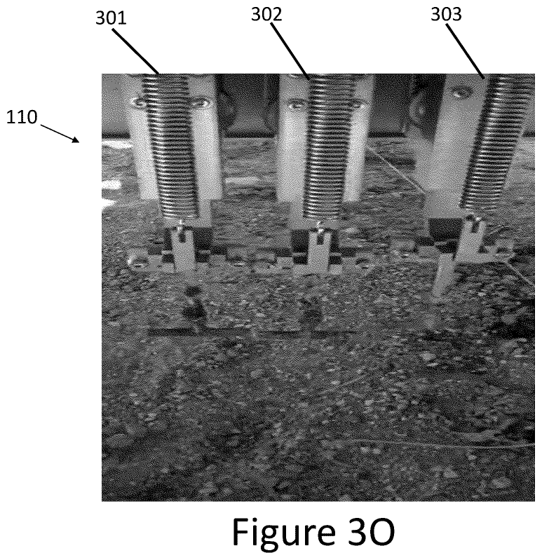

FIG. 3A shows a schematic illustration of the mechanical module 110, in accordance with embodiments. The mechanical module 110 is configured and enabled to provide tilling at varying ground penetration depths, for example, based on the received Instruction Signals 216. In an embodiment, the mechanical module 110 comprises one or more rows, each row comprises a plurality of implements arranged side by side wherein each implement, such as implement 360, covers a given width across the width of the mechanical module 110. As an example, each implement 360 may cover 5 cm or less. The specific design, shape and size of each implement and the number of implements and rows may be varied according to the system's design the nature of the soil, the plot arrangement and the agricultural action. In some embodiments, the mechanical module 110 may comprise a single or only two implements.

In an embodiment, the mechanical module 110 comprises a weeding control module 330 and/or a communication circuitry 340 capable of receiving instructions from the control module 250 for executing a localize adaptive tilling action.

Each or some of the implements comprise a controller 362. The controller 362 is an electronic driver configured and enabled to receive instruction signals from the Weeding Control module 330 and/or directly from the processor(s) 240 and accordingly control the implement's' motor speed/direction according to the location and amount of the identified weeds/crop.

In an embodiment, the operator might install different end-effectors for intra-row and inter-row operation. In an embodiment, the operator might install different implement's tools for different agricultural conditions such as soil type, crop type, time of year, and the like. In an embodiment, the system can automatically select the appropriate implement/end effector type and apply the right end effector type, shown in FIG. 3J-FIG. 3M, according to inputs from the sensing module 120 and according to some pre-defined logic.

In an embodiment, as shown in FIG. 3A, the mechanical module 110 comprises more than one row of implements, for example, two rows of implements 310 and 320. In an embodiment, the rows may be staggered with respect to one another such that the center location of each implement of the 2^{nd} row 320 is located to cover the small gap between adjacent implements of the 1^{st} row 310.

FIG. 3B shows a top-left-side three-dimensional illustration of the mechanical module 110 including two rows of implements, wherein each implement 360 is capable of moving up and down in parallel to axis Z. As shown in FIG. 3B the plurality of implements in the second row 320 slightly and partially overlap the first row 310 in a ‘zipper’ design view.

FIG. 3C shows a slightly tilted top three-dimensional illustration of the mechanical module 110, in accordance with embodiments. The mechanical module 110, comprises two rows 310 and 320 of implements setup wherein each implement 360 is capable of conducting vertical movement up and down, independently and/or autonomously in parallel to axis Z (e.g. and perpendicular to the movement Y direction of the vehicle holding the mechanical module 110), in accordance with embodiments. Specifically, as illustrated in FIG. 3D an implement 360 includes an upper section body 365 for housing a piston or a motor (e.g. included for example inside the upper section) for providing vertical motion up/down of the implement with respect to the implement movement (e.g. along x axis) and/or Force limiting springs that can produce vertical movement up and down for lowering an end effector 370 of the implement 360 into the ground (e.g. soil 190) as needed based on the weeding strategy instructions signals. The piston or motor may be one or more of hydraulic, pneumatic or electrical, and/or include any mechanism to enable vertical movement of the implement 360, in accordance with embodiments.

FIG. 3N shows a perspective view 307 of the mechanical module 110, which comprises two rows 310 and 320 of implements arranged in a stacked staggered array, in accordance with embodiments.

FIG. 3O shows an image of the mechanical module 110 comprising three implements 301, 302, 303 each comprising end effector comprising a rod such as a scalpel shape rod as shown in FIG. 3L, in accordance with embodiments.

In another embodiment, the implement 360 may be based on a lever mechanism.

In another embodiment, the implement 360 may be based on a spiral screw mechanism.

In an embodiment, the mechanism providing the vertical motion may be split into two mechanisms such as two separate mechanisms one capable of operating slow motion and one capable of fast motion. In some embodiments, the slow motion may be up to 500 mm/sec, typically 100 mm/sec and the fast motion above 500 mm/sec, typically 800-1000 mm/sec, although it should be noted that other speeds are possible.

The slow motion may be used to adjust the height of the implements above ground (e.g. soil 190) in order to follow the ground structure (e.g. terrain following movement), while the fast motion may be used to conduct the tilling action. With such an embodiment it may be desired to have the slow motion of several implements joined so they will move together as a single block, while still allowing the fast tilling motion of each implement to be separated. This operation method offers an efficient design utilizing the fact that the terrain following may not require high velocity vertical motion as the tilling action.

In accordance with some embodiments, the end effector 370 of the implement may be one or more of a: a blade, a rod, a wire, a comb-like structure, etc.

FIG. 3J-FIG. 3M show examples of various types of end effectors 390 shapes that may be connected or embedded to the edge of the implement 360, in accordance with embodiments.

In accordance with embodiments, each end effector is connectable and detachable utilizing connection mechanisms well-known in the field.

FIG. 3J shows a perspective top-side view 391 and a side view 392 of a ‘fork’ shaped end effector 392′ comprising two fork blades 388 and 389 located at the sides of the end effector. A blade 391′ shape may be elongated narrow rectangular shape for easily penetrating and tiling weed, in accordance with embodiments. A thin metallic wire may be attached at the bottom between the fork's side in order to provide the tilling/weeding action.

FIG. 3K shows a perspective top-side view 394 and side view 393 of an end effector 394′ having a comb shape comprising for example seven blades 393′ configured and enabled to till the soil, for example at a dense weed soil area. A single blade's 393′ shape of the comb may have a wide upper section that tapers to a narrow sharp end.

FIG. 3L shows a perspective top side view 396 and a side view 395 of an end effector 396′ comprising a rod such as a scalpel shape rod 395′ connected at the edge of a stick 399, in accordance with embodiments.

FIG. 3M shows a perspective top side view 398 and a side view 397 of an end effector 398′ having a rectangular frame structure. The frame shape and size at the sides of the rectangular may have an elongated shape 397′ as illustrated in FIG. 3M. An example of the end effector having a rectangular shape is also illustrated in FIG. 3F.

In some cases, the end effector, such as end effector 370 shown in FIG. 3E can also include one or more moving parts such as moving blades or a moving saw chain that would allow grinding plants and the soil. The surface of the end effector 370 may be made rough (e.g. by sand spray) in order to increase the friction with the plant material allowing better weeding. Alternatively or in addition, the end effector as illustrated in FIG. 3K may include a comb-like structure in order to allow better attachment of plant material. In a preferred embodiment, the end effector 370 can be used by both to cut through the ground and to cut plant leaves above ground. The end effector 370 can also be or include a non-mechanical elements such as a heat source, electrical discharge source, liquid spraying nozzle, air blower, etc.

FIG. 3E shows an illustration of an end effector 370, in accordance with embodiments. The end effector 370 comprises a single horizontal rod 372 connected at the bottom distal end of an elongated holder 374 for efficiently penetrating the ground and tilling the ground and the plants, in accordance with embodiments. The implement further comprises element 376 for connecting the end effector 370 to and/or into the upper section body 365 of an agricultural implement such as implement 360.

FIG. 3F shows an additional optional design of an end effector 390 comprising a rectangular structure 382, and respectively FIG. 3G shows an end effector 390 including a blade 392 or a thin wire embedded in the rectangular structure, in accordance with embodiments. The blade 392 acts like a kitchen potato peeler. The lower part passes through the soil and root and removes the weed. The end effector shape and/or design is optimized, in accordance with embodiments, to allow the best efficiency in penetrating the ground and tilling the ground and the plants. The end effector shape and design may be interchangeable for different soil types and vegetation states according to the agricultural date and weeding strategy instructions for adaptively and selectively tilling the soil.

In an embodiment, the holding structure (e.g. single arm as shown in FIG. 3E vs. rectangular frame as shown in FIG. 3F and FIG. 3G) is optimized to allow the best rigidity and structure.

In a preferred embodiment, the vertical motion of the implement may take place while the vehicle (e.g. tractor 180) is moving forward in the field. To produce minimal tilling and prevent crop damage the vertical motion of the implement typically needs to be comparable in speed or faster than the forward motion of the entire weeding vehicle (e.g. tractor 180).

The vertical motion of each of the implements may be controlled by controller 362. The controller 362 is an electronic driver configured to receive/send the instruction signals to the implement's motor and control the magnitude and the speed of the vertical motion based on inputs from the processor(s) 240 of the processing module 230. As an example, processor 240 may determine, based on the sensory data analyses, that a specific weed observed by the sensing module 120 requires weeding at a depth of for example 2 cm below ground. The processor(s) 240 will then instruct the controller 362, for example via the weeding control module 330, of the appropriate implement to execute the appropriate vertical motion to ensure ground penetration at the right location to the desired depth of 2 cm.

In an embodiment, the result of the action of the implement may be a ground penetration at a footprint of for example 5×5 cm, with the width of 5 cm coming from the size of the implement end effector, and the length of 5 cm coming from the duration in which the implement stays below ground, coupled with the forward motion of the whole system.

In an embodiment, the mechanical module 110 includes a mechanism that allows forward motion compensation (FMC) to allow momentary vertical motion in the world of the implements. Such a mechanism would move each implement slightly backward while the mechanical module 110 keeps moving forward (parallel to axis Y). The result of the backward movement will be that the vertical motion (parallel to axis Z) would follow a steeper curve with respect to the field (e.g. the soil), which may create a preferable ground penetration profile and a smaller longitudinal tilling area. As an example, when the FMC mechanism is configured and enabled such that the backward movement will temporarily compensate completely the forward motion of the vehicle the result will be that each implement is conducting a completely vertical only motion in the exact place with respect to the ground, while the vehicle is moving forward in the field.

In an embodiment, the implements may be equipped with a force limiter mechanism. Such a mechanism would limit either or both of the vertical and horizontal forces that can be extracted on each implement. This may be done to prevent mechanical damage and failure of the implements in case a sturdy obstacle is encountered. Instead of having the implement braking or failing, the force limiter will make the implement recoil back.

In an embodiment, the rows of implements may be mounted on rails 363 or a similar mechanism allowing lateral adjustment perpendicular to the direction of motion of the implement. This configuration provides better alignment of each implement according to the location of the field's crop rows.

In an embodiment, each or some of the implements 360 may also be equipped with a force gauge sensor 364, allowing detection when the implement has penetrated the ground. Such a scheme help in timing the vertical motion of the implement correctly to induce the desired minimal tilling. This may also help to overcome some of the tolerances and inaccuracies of the sensing module 110 3D localization function.

FIG. 3H and FIG. 3I show respectively a cross-section view and a perspective side view of implement 361, in accordance with embodiments. The implement 361 comprises an engine, for example, a rotor engine 350 for rotating a strap 351 and enabling the vertical movement (parallel to axis Z) of the implement body 365 along a first track 352 holding the implement body 365. The implement further comprises a first spring 354 connected to the end effector 370 and to a second track 653, wherein both are housed in the implement body 365. The spring 354 is in a loaded state, and when the end effector 370 encounters a hard object in the ground, the Spring 354 collapses, absorbing the impact along with the end effector 370 to prevent it from breaking and as a result the end effector 370 is folded inside into the implement body 365.

The implement 361 further comprises, in accordance with embodiments, a second spring 355 located at the bottom distal end of the implement 361, and connected to the end effector 370. The second spring 355 is needed for cases where the end effector 370 penetrates the ground and hits a hard element, such as a stone. As a result of the collision with the hard element, the second spring 355 will cause the end effector 370 to fold upwards, parallel to the Z-axis and against the direction of the implement's movement.

In operation, the first track 352 lowers repeatedly the end effector 370 up and down, parallel to Z axis, for tilling weeds. In case the weeds and crop are soft, the end effector including the respective tools (blade, a rod, a wire, a comb-like structure, etc.) will till the weeds. However, if the end effector 370 hits a hard element, for example above the soil, which is stronger than the spring 354 power, it will automatically fold inward using the spring 354 and second track 653 into the implement body, similar to how the blade of a switchblade folds into its handle. In case the end effector 370 penetrates the ground and hits a hard element, such as a stone in the ground which is stronger than the second spring 355 power, the implement 361 and/or the end effector 370 will fold backward, parallel to the X-axis and against the direction of the implement's movement direction. In accordance with embodiments, the first spring 354 and the second spring 355 power may be adjusted according to the soil/crop/weed type.

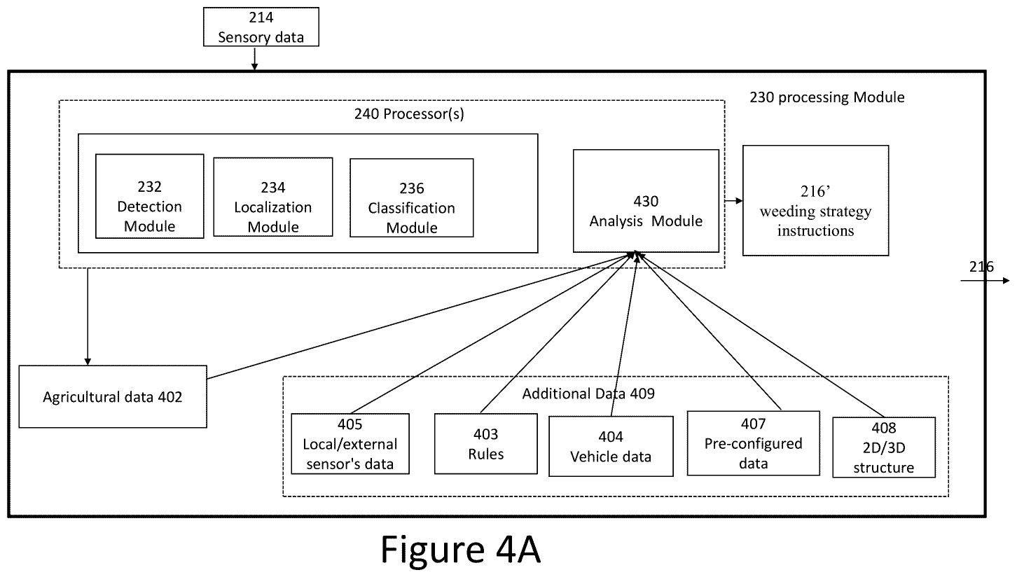

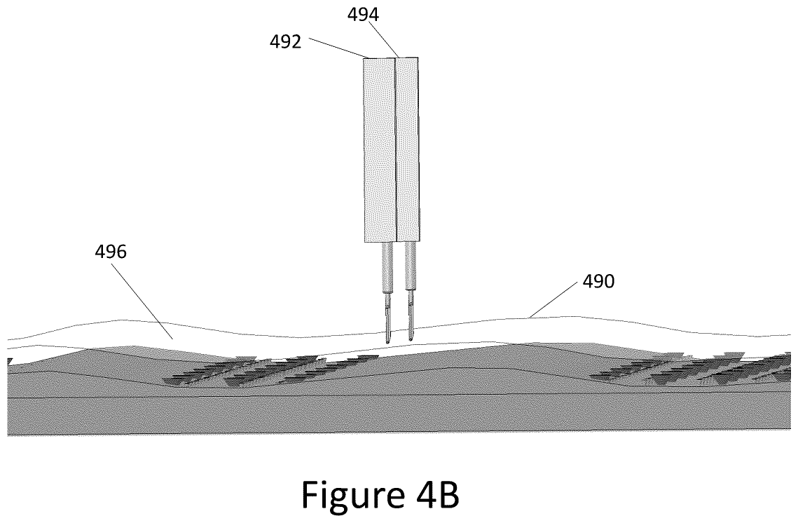

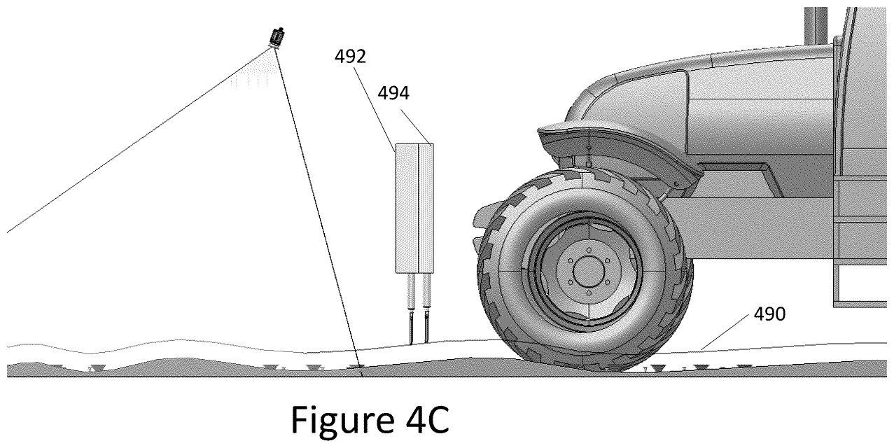

FIG. 4A shows a schematic detailed block diagram of the processing module 230, in accordance with embodiments. The processing module 230 is configured and enabled to receive sensory data 214 from the sensing module 120, process and analyze the agricultural data 402 and additional data 409 to yield weeding strategy instruction signals 216 for instructing the Mechanical module 110 on the weeding action.

In some embodiments, only the agricultural data 214 is used to yield the weeding strategy instruction signals 216.

The additional data 409 may comprise one or more of: local/external sensor's data 405, rules 403 (e.g. predefined set of rules), vehicle data 404, pre-configured data 407 and 2D/3D structure 408.

In accordance with embodiments, some or all data of the additional data 409 such as the pre-configured data 407 which is known in advance, may be uploaded automatically or manually by the user (e.g. the farmer) to the processing module 230, using for example the user's mobile device (e.g. smart phone).

In accordance with embodiments, the pre-configured data 407 may include one or more of: soil type (e.g. clay, sandy, silty, loamy, peaty, chalky, saline) crop type, weather, type/model of the vehicle (e.g. tractor).

The 2D/3D structure 408 may include 2D/3D structure of scene 201. In some cases, the 2D/3D structure 408 may be based on the Sensory Data 214 and/or on external data such as 2D/3D images received from external sensors.

The additional data 409 and the agricultural data 402 are transmitted to the analysis module 430 which analyzes the received data to yield weeding strategy instructions 216′.

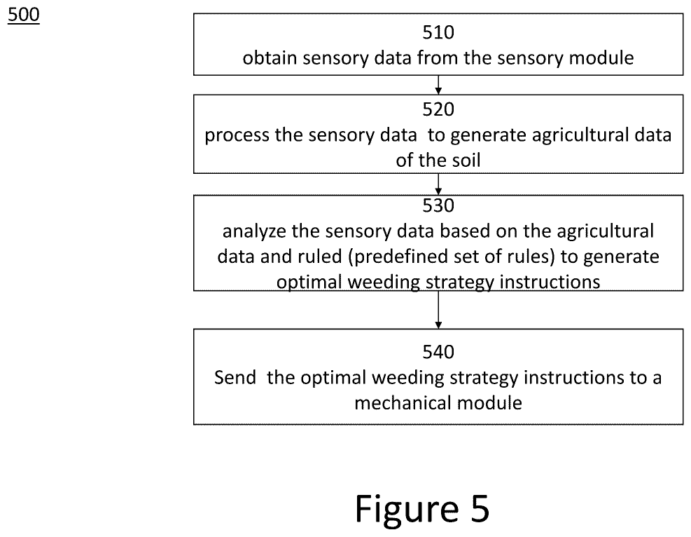

As an example, the processing module 230 may execute the following steps as illustrated in flowchart 500 of FIG. 5, flowchart 600 of FIG. 6A and flowchart 605 of FIG. 6B.

In an embodiment, in order to correctly and precisely execute the tilling/weeding, system 100 needs to know the location of the weeds detected by the sensing module 110 with respect to the weeding implements 360. In some cases, it is preferred to have the sensing module 110 sense an area slightly in front of the mechanical module 110, as to allow enough time for the processor(s) 240 to complete its processing. In such a setup a 3D odometry mechanism may be employed in order to obtain the 3D displacement between the weeds' location with respect to the sensing module 120 and the weeds' location with respect to the mechanical module 110 after the system (e.g. vehicle-tractor 180) had traveled forward. Such 3D odometry may be obtained, as an example, by either of the following methods:
1. Assuming straight line motion and using time and speed;
2. Measuring the motion by an encoder attached to the system support wheels or to a dedicated wheel.
3. Using an exact navigation system such as either RTK GPS, an IMU or a combination of both;
4. Using visual odometry, i.e. visually matching the images from the sensing module in order to continuously calculate the 3D displacement of the Mechanical module 110 in space.

FIG. 4B and FIG. 4C show an exemplary illustration of two implements 492 and 494 following the terrain profile lines 490 to ensure optimal tilling action when needed, in accordance with embodiments. The semi-transparent surface 496 is a virtual surface representing the optimal terrain following position as inferred by the sensing module 110, in accordance with embodiments. The semi-transparent surface 496 is generated based on 3D images and depth maps of scene 201, such as 3D image 603. The thin profile lines 490 above the ground are generated by the processing module and follow the terrain at a fixed elevation above the ground. In operation, the tip of the implements are kept along this line. It is clarified that the elevation above the ground of the terrain following is configurable. A higher position would allow larger clearance above the ground and would fit a stage when the crop plants are higher. As an example, if the crop is at a stage it can reach 20 cm the terrain following would be pre-set to be at 25 cm as to not harm the crop. A lower position would allow a shorter time to reach the ground level when tilling a weed, hence allowing a faster action time and therefore treating a larger number of weeds per a given forward driving speed.

FIG. 5 illustrates flowchart 500 of a method for advanced adaptive and selective agricultural weeding and/or tilling, using one or more sensors such as imaging devices, in accordance with embodiments. In other embodiments, the method 500 includes different or additional steps than those described in conjunction with FIG. 5. Additionally, in various embodiments, steps of the method may be performed in different orders than the order described in conjunction with FIG. 5.

System 100 or one or more processors such as processor(s) 240, for example, may be used to implement method 500. However, method 500 may also be implemented by systems or processors having other configurations. In other embodiments, the method includes different or additional steps than those described in conjunction with FIG. 5. Additionally, in various embodiments, steps of the method may be performed in different orders than the order described in conjunction with FIG. 5.

At step 510 sensory data 214 are obtained from the sensory module 120, the sensory data 214 may include for example one or more of: 2D or 3D images, for example a sequence of 2D or 3D visual images of the soil 190 as illustrated for example in FIGS. 7A-7E.

At step 520 the sensory data 214 is processed and analyzed by the processing module 230 to generate agricultural data 402 related to the soil 190 which needs to be tilled/weed. The agricultural data 402 may include one or more of: crop and/or weeds type, growth stage and location information from the sensing module for the current field of regard, such as location of weed in the soil with respect to crop, weed size, weed stage, soil 3D structure, soil type, terrain structure (e.g. terrain structure of the scene/soil), geometrical data of the scene, 3D structure of the scene.

At step 530 the agricultural data 402 and additional data 409 are further analyzed, using for example the analysis module 430, to yield optimal weeding strategy instructions and actions 216′

In accordance with embodiments, the optimal weeding strategy instructions and actions 216′ may include one or more of the following examples:
a. Avoid removing too small weeds that cannot harm the crop since the crop will outgrow them;
b. Avoid removing too large perennial weeds since they cannot be effectively removed;
c. Avoid removing weeds too close to crop;
d. Avoid rocks and other obstacles;
e. Till at soil level or at a shallow depth for broadleaves weeds; and
f. Till at a larger depth for grass-like weeds and for large weeds.

In accordance with emblements, the vehicle data 404 comprise one or more of: the speed, location and/or motion (e.g. motion direction) and/or speed (e.g. forward motion) of the vehicle holding the mechanical module.

In accordance with embodiments, the local/external sensor's data 405 comprise one or more of data obtained from sensors such as RTK (real time kinematics) GPS (Global Positioning System), an IMU (Inertial Measurement Unit) data, a wheel encoder.

In accordance with embodiments, the rules 403 may include one or more of the following examples:
a. The action (e.g. the mechanical tilling/weeding action) of the mechanical module should be selective and match to the obtained weed type, stage and location with appropriate tilling size and depth. Specifically, the identified location of crop is used to prevent tilling action that would endanger the crop. The result of such selective action is a strategically tilling action based on the location and/or depth of tilling action for each detected weed.
b. The action (e.g. the mechanical tilling/weeding action) of the mechanical module should be based on the obtained optimal terrain structure (e.g. 3D terrain structure) to adjust the elevation of each implement above ground that would allow optimal tilling/weeding. Specifically, the detected terrain structure is used to perform the action at the right location and depth. Without knowing the 3D structure, it is impossible to perform a localized tilling/weeding action at the right location and at the right depth.
c. Limit the simultaneous tilling action in order to prevent harm to the machine or to optimize power consumption and efficiency. This is achieved based on the identified Terrain structure which is used to enable each action unit (e.g. implement 360) hover above ground at a fixed height. This action is performed in order to allow clearance from crop while minimizing the action time (in other words, hovering the mechanical module higher than needed would mean that it takes more time to drop for the action, reducing the number of actions per second that can be done. Hovering lower would mean that there is not enough room to pass above the crop).
d. Prioritize weeding importance in case the limit above does not allow weeding of all the weeds.
e. Monitor the location and/or motion (e.g. motion direction) and/or speed (e.g. forward motion) of the system 100 (e.g. the mechanical module 110 or the vehicle 180) in order to time/speed correctly synchronize the tilling action of the implements with the velocity/rate of the vehicle. This may be achieved based on the captured sensory data 214 and alternatively or additionally supported by other modules such as wheels encoder and/or GPS-RTK.

In accordance with embodiments, the sensory data 214/agricultural data 402 is further analyzed to generate strategy logic optimizing and prioritizing the desired action within the mechanical module 110 and/or the vehicle (e.g. tractor 180) constraints. Some examples of such constraints include one or more of:
Number of action units working simultaneously; and
Coping with high density of weeds.

At step 540 the optimal weeding/tilling strategy instructions 216′ are transmitted as instructions signals 216 to the mechanical module 110. Specifically, the instructions are transmitted to the implements' controllers 362 in order to conduct the tilling/weeding action. In an embodiment, the strategy instructions 216′ are converted into instruction signals 216 using established electronic conversion methods.

FIG. 6A illustrates a flowchart of a method 600 for advanced adaptive and selective agricultural weeding and/or tilling, using a single imaging device such as a single camera, for example a 5 MP RGB camera, in accordance with embodiments. In other embodiments, the method 600 includes different or additional steps than those described in conjunction with FIG. 6A. Additionally, in various embodiments, steps of the method may be performed in different orders than the order described in conjunction with FIG. 6A.

Some or all stages of method 600 may be carried out at least partially by at least one computer processor, e.g., by processor(s) 240. Respective computer program products may be provided, which comprise a computer-readable storage medium having computer readable program embodied therewith and configured to carry out of the relevant stages of method 600. In other embodiments, the method includes different or additional steps than those described in conjunction with FIG. 6A. In some embodiments, some of the steps of the method are optional, such as the filtering process.

At step 605 a new visual image, for example, a 2D images, of the scene 201 captured by the imager 223 is obtained while a previous image captured by the imager 223 is stored, for example at the Storage/Memory Device 254, in accordance with embodiments. The imager 223 may be for example one or more imagers 112, 114, 116 shown in FIG. 1A. Specifically, the imager may be a camera such as RGB camera. The obtained images (e.g. a sequence of new and previous 2D images) are captured and/or processed, for example in real-time or close to real-time as the vehicle (tractor 180) drives in the scene 201. The obtained sequence of 2D images include images of the scene 201 including images of the crop and for example images of various types of weeds. In accordance with some embodiments, the 2D images are captured synchronically and/or sequentially by the camera located for example at the front or back section of the vehicle as illustrated in FIG. 1A. An example of an original captured images 601 is shown in FIG. 6B. Image 601 includes an image of the soil 190 including images of the crop 205 and weeds such as Broad-leaves weed-202.

At step 610, a depth map of the scene 201 is created from the new image and the previous image using for example traditional stereo vision techniques for modeling the scene. These techniques include using corresponding points in both images to determine relative disparities, which are then used to compute depth information of the scene. In some cases, image processing methods may be applied for accuracy, including sub-pixel interpolation, occlusion handling, and filtering. An example of a depth map image 702 created from the original image 701 of FIG. 7A is shown in FIG. 7B. The bright sections 711 of the depth map image 602 relates to larger distances from the camera, therefore lower points on the soil since the camera is viewing the area from above, and the dark sections 713 relates to smaller distance from the camera and hence higher points on the soil. FIG. 7C shows an example of a perspective depth map image 703 created based on the original image 701.

At step 615 one or more algorithms such as neural network algorithms are applied on the obtained new image to create a segmentation map, in accordance with embodiments.

For example, a machine learning neural network, in accordance with embodiments, is trained on prior images to mark and identify in the images (e.g. included in the sensory data 214) the identified plants. The machine learning is conducted by feeding annotated examples of images in which the plants are marked as desired. The marking can be one or more of:
Classification—The entire fed image is marked according to its contact;
Bounding box—Object of interest are marked by a bounding box with the appropriate label; and
Segmentation—A mask is produced in which all the image pixels which are part of the object arc labeled.

FIG. 7D shows an example of an image 714 of a Patch of ground 706 imaged by the camera and FIG. 7E shows an example of corresponding segmentation map 715 of plants 708 (the bright dots) and soil 707 detected by the neural network, in accordance with embodiments.

At step 620 the segmentation map and the depth map are merged to yield the agricultural data 402 comprising one or more of plants and/or weed detection and/or classification and/or 3D location of the detected plants and/or weed, in accordance with embodiments.

At step 625 the agricultural data 402 (e.g. including the obtained 2D/3D location of the weed and/or crop, type) and the additional data 409 are processed and/or analyzed, using one or more processors, such as an analysis module 430, to yield weeding and/or tilling actions (e.g. weeding strategy instructions 216′). Specifically, the processing step comprises calculating the specific weeding/tilling action relating to the imaged soil. The specific weeding/tilling actions relate to when (time), where (location), and how (speed/rate) perform the weeding/tilling action. In an embodiment, the processing further includes encoding the instructions to generate instructions signals 216 to be sent to the mechanical module 110.

Optionally, in some embodiments at step 630 the terrain following profile for the mechanical module path is extracted from the depth map(s). An example of the extracted terrain profile lines 490 is illustrated in FIG. 4A and FIG. 4B.

At step 635 the instructions signals (e.g. instruction signals 216), comprising the weeding and/or tilling actions (e.g. when and/or where and/or how to perform a tilling action), are transmitted to the mechanical module 110 for accordingly operating the instructions. Specifically, the instruction signals are sent from the control module 250 to the one or more controllers 362 of each respective implement to operate the tilling process. More specifically, based on the instructions the controller may control the magnitude and/or the speed of the vertical motion of the mechanical module (e.g. the vertical motion of the implements, such as implement 360).

FIG. 6B illustrates a flowchart of a method 606 for advanced adaptive and selective agricultural weeding and/or tilling, using a stereoscopic imaging device such as a stereoscopic camera, in accordance with embodiments. In other embodiments, the method 606 includes different or additional steps than those described in conjunction with FIG. 6B. Additionally, in various embodiments, steps of the method may be performed in different orders than the order described in conjunction with FIG. 6A.

Method 606 present all steps of the aforementioned method 600 but instead of step 605 includes at step 640 using a stereoscopic imager such as a stereoscopic camera.

At step 640 a new 3D image such as a stereo image of the scene 201 captured by the stereoscopic camera is obtained, in accordance with embodiments. The obtained images are captured and/or processed, for example in real-time or close to real-time as the vehicle (e.g. tractor 180) drives in the scene. The obtained sequence of stereo images includes images of scene 201 including images of the crop and for example various types of weeds. In accordance with some embodiments, the stereo images are captured sequentially by one or more stereo cameras located for example outside the vehicle's cabin, for example at the front or back section of the vehicle as illustrated in FIG. 1A.

At step 645, a depth map of the scene is created from the stereo image using for example traditional stereo vision techniques. An example of a depth map image 702 created from the original image 701 is shown in FIG. 7B. The bright sections 711 of the depth map image 702 relates to larger distances from the camera, therefore lower points on the soil since the camera is viewing the area from above, and the dark sections 713 relates to smaller distance from the camera and hence higher points on the soil. FIG. 7C shows an example of a perspective depth map image 703 created based on the original image 602.

At step 650 one or more algorithms such as neural network algorithms are applied on the obtained new image images to create a segmentation map, in accordance with embodiments.

For example, a machine learning neural network, in accordance with embodiments, is trained on prior images to mark and identify in the images (e.g. included in the sensory data 214) the identified plants. The machine learning is conducted by feeding annotated examples of images in which the plants are marked as desired. The marking can be one or more of:
Classification—The entire fed image is marked according to its contact;
Bounding box—Object of interest are marked by a bounding box with the appropriate label; and
Segmentation—A mask is produced in which all the image pixels which are part of the object are labeled.
   FIG. 7D shows an example of image 714 of a Patch of ground 706 imaged by the camera and FIG. 7E shows an example of corresponding segmentation map 715 of plants 708 (the bright dots) and soil 707 detected by a neural network, in accordance with embodiments.
   At step 655 the segmentation map and the depth map are merged to yield agricultural data 402 comprising one or more plants and/or weed detection and/or classification and/or 3D location of the detected plants and/or weed, in accordance with embodiments.

At step 660 the agricultural data 402 (e.g. including the obtained 2D/3D location of the weed and/or crop, type) and the additional data 409 are processed and/or analyzed to yield weeding and/or tilling actions. Specifically, the processing and/or analyzing step comprises calculating the specific weeding/tilling action relating to the imaged soil. The specific weeding/tilling actions relate to when (Time), where (location), and how (speed/rate) perform the weeding/tilling action. In an embodiment, the processing further includes encoding the instructions to generate instructions signals 216 to be sent to the mechanical module 110.

Optionally, in some embodiments at step 665 the terrain following profile for the mechanical module path is extracted from the depth map(s). An example of the extracted terrain profile lines 490 is illustrated in FIG. 4A and FIG. 4B.

At step 670 the instructions signals, comprising the weeding and/or tilling actions (e.g. when and/or where and/or how to perform a tilling action), are sent to the mechanical module for accordingly operating the instructions. Specifically, the instruction signals are sent from the control module 250 to the one or more controllers 362 of each respective implement to operate the tilling process. More specifically, based on the instructions the controller may control the magnitude and the speed of the vertical motion of the mechanical module (e.g. the vertical motion of the implements, such as implement 360).

In an embodiment, the instruction signals further comprise terrain follow instruction which are sent to the mechanical module to follow the identified terrain line (e.g. terrain line 490). Specifically, the instruction signals comprise weeding and/or tilling actions as well as information of the terrain follow of the soil. In an embodiment, the instructions comprising the information of the terrain follow of the soil are sent to the one or more controllers 362 for activating accordingly the related implement.

In further embodiments, the processing module may be a digital processing device including one or more hardware central processing units (CPU) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

The processor(s) and/or processing module in the present patent application encompasses various embodiments, including but not limited to edge processing or stand-alone processing or embedded system with the processor(s) onboard utilizing advanced technologies such as NVIDIA, Intel, AMD, Qualcomm, and ARM, chips, as well as other cutting-edge chips from leading companies in the industry. These chips can be utilized within the processor(s) and/or processing module.

The above-described systems and method can be executed by computer program instructions that may also be stored in a computer-readable medium or a dedicated embedded device such as a chip that can direct a computer, other programmable data processing apparatus, or other devices to function in a particular manner, such that the instructions stored in the computer-readable medium produce instructions which when implemented cause the writing assistant to perform the above-described methods.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the above-described methods.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered.

In some embodiments, the system disclosed herein includes software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

In some embodiments, the system disclosed herein includes one or more databases, or use of the same. In view of the disclosure provided herein, those of skill in the art will recognize that many databases are suitable for storage and retrieval of information as described herein.

In the above description, an embodiment is an example or implementation of the inventions. The various appearances of “one embodiment,” “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is to be understood that the phraseology and terminology employed herein is not to be construed as limiting and are for descriptive purpose only.

The principles and uses of the teachings of the present invention may be better understood with reference to the accompanying description, figures and examples.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element. It is to be understood that where the specification states that a component, feature, structure, or characteristic “may”, “might”, “can” or “could” be included, that particular component, feature, structure, or characteristic is not required to be included. Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described. Methods of the present invention may be implemented by performing or completing manually, automatically, or a combination thereof, selected steps or tasks.

The descriptions, examples, methods and materials presented in the claims and the specification are not to be construed as limiting but rather as illustrative only. Meanings of technical and scientific terms used herein are to be commonly understood as by one of ordinary skill in the art to which the invention belongs, unless otherwise defined. The present invention may be implemented in the testing or practice with methods and materials equivalent or similar to those described herein.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A system for adaptively and selectively tilling soil for agricultural weeding, the system comprising:
a sensing module comprising at least one sensor configured and enabled to capture sensory data of the soil;
a mechanical module comprising at least one implement, wherein said at least one implement is configured and enabled to perform tilling in the soil;
a control module comprising:
a communication circuitry for communicating with said sensing module and said mechanical module; and
a processing module, wherein said processing module comprising:
one or more processors, wherein said one or more processors are configured and enabled to:
process and analyze the captured sensory data to generate agricultural data of the soil;
analyze the agricultural data and additional data to yield weeding strategy instruction signals; and
transmit the weeding strategy instructions signals to the mechanical module for adaptively and selectively till or weed the soil.

2. The system of claim 1, wherein the processing module comprises:
a detection module configured and enabled to analyze the sensory data to mark and discriminate plants from non-plants in said soil;
a classification module configured and enabled to analyze the sensory data to distinguish different plants type in said soil; and
a localization module configured and enabled to analyze the sensory data to identify the location of a plant's elements in the soil.

3. The system of claim 2, wherein the detection module or classification module are based on computer vision algorithms utilizing shape or color features.

4. The system of claim 2, wherein the detection module or classification module are based on one or more machine learning algorithms.

5. The system of claim 4, wherein the one or more machine learning algorithms are trained using labeled data.

6. The system of claim 4, wherein the one or more machine learning algorithms are based on deep learning algorithms, wherein said deep learning algorithms utilizing neural networks.

7. The system of claim 1, wherein the sensing module comprises at least one imager for capturing one or more images of the soil or a scene comprising the soil.

8. The system of claim 1, wherein the sensing module comprises an illumination module said illumination module comprises at least one illumination source.

9. The system of claim 1, wherein the sensing module is configured and enabled to construct a 2D or 3D model of the soil or scene.

10. The system of claim 7, wherein said at least one imager is selected from the group consisting of:
an RGB camera, a monochrome camera, a thermal camera, a multi-spectral camera, a stereo camera, Time of Flight sensor, LIDAR sensor, RF sensor.

11. The system of claim 1, wherein the sensory data comprises one or more of: 2D or 3D images, and wherein a 2D or 3D model of the soil or scene is constructed based on said 2D or 3D images.

12. The system of claim 1, wherein the sensing module comprises at least two imagers, each imager having a predefined image capturing area and wherein there is a predefined overlap between the captured areas of the at least two imagers.

13. The system of claim 1, comprising an additional sensing module configured and enabled to monitor the soil following the tilling action to provide quality assurance.

14. The system of claim 1, wherein the agricultural data comprises one or more of:
crop or weeds type, growth stage, 2D or 3D location information of the crop or weeds, geometrical data, 3D structure of the scene.

15. The system of claim 1, wherein the mechanical module comprises an end effector.

16. The system of claim 1, where the tilling action is conducted at a varying penetration depth.

17. The system of claim 1, wherein said at least one implement comprises:
an upper section body for housing a motor said motor is configured and enabled to provide vertical motion of the implement with respect to the implement movement;
at least one spring configured and enabled to lower the end effector into the soil based on the weeding strategy instructions signals.

18. The system of claim 17 wherein the motor is configured and enabled to rotate a strap for enabling the vertical movement of the implement's body along a first track.

19. The system of claim 18, wherein said at least one implement further comprises:
a first spring connected to the end effector and to a second track, wherein said first spring is in a loaded state, and wherein the first spring is configured to vertically collapse for absorbing the impact along with the end effector to prevent it from breaking.

20. The system of claim 18, wherein said at least one implement further comprises:
a second spring located at the bottom distal end of the implement and connected to the end effector, said second spring is configured to cause the end effector to fold upwards, parallel to the direction of the implement's movement.

21. The system of claim 15, wherein said mechanical module comprises:
at least one row of implements, wherein said implements are arranged side by side, and wherein each implement of said implements covers a given width across the width of the mechanical module, and wherein each implement of said implements is configured and enabled to move up or down with respect to the movement of said mechanical module.

22. The system of claim 21, wherein the one or more processors are configured and enabled to:
process the captured sensory data to extract a terrain profile of the soil.

23. The system of claim 22, wherein the mechanical module comprises at least two implements configured and enabled to follow the terrain profile of the soil to ensure optimal tilling action of the soil.

24. The system of claim 17, wherein the vertical motion of the at least one implement is split into two separate mechanisms a first mechanism capable of operating slow motion of up to 500 mm/sec and a second mechanism capable of fast motion in the range 800-1000 mm/sec.

25. The system of claim 24, wherein the slow motion of the at least one implement is configured and enabled to adjust the height of the at least one implement above the soil and to follow said extracted terrain following movement.

26. The system of claim 24, wherein the fast motion is configured to conduct a tilling action.

27. The system of claim 24, wherein in the slow motion two or more implements are joined whereas in the fast motion, each implement moves vertically separately.

28. The system of claim 1, wherein the mechanical module comprises a mechanism that allows forward motion compensation (FMC).

29. The system of claim 1, wherein the mechanical module comprises force limiters.

30. The system of claim 15, where the end effector comprises one or more of:
a blade, a rod, a moving blade, a saw.

31. The system of claim 1, wherein the mechanical module comprises a force gauge.

32. The system of claim 1, wherein the additional data comprises one or more of:
rules, vehicle's data, pre-configured data, 2D or 3D structure, local or external sensor's data

33. The system of claim 32, wherein said rules include one or more of:
a. Match each weed type stage and location in said soil with appropriate tilling size and depth;
b. Use location of crop in said soil to prevent tilling action that would endanger the crop;
c. Obtain an optimal terrain following the elevation of each implement above ground that would allow optimal tilling;
d. Limit the simultaneous tilling action in order to prevent harm to the mechanical module or to optimize power consumption and efficiency;
e. Prioritize weeding importance in case the limit above does not allow weeding of all the weeds;
f. Monitor the location of the system and its forward motion in order to time correctly the tilling action of the at least one implement.

34. The system of claim 1, wherein the weeding strategy instructions signals comprise one or more of the following instructions:
avoid removing too small weeds that cannot harm the crop; avoid removing too large perennial weeds; avoid removing weeds too close to crop; avoid rocks and other obstacles;
till at soil level or at a shallow depth for broadleaves weeds; till at a larger depth for grass-like weeds and for large weeds.

35. The system of claim 1, comprising a storage unit for storing said sensory data or additional data.

36. The system of claim 1, wherein the mechanical module is towed or shoved by a vehicle, said vehicle is selected from the group consisting of:
an autonomous vehicle, a tractor, a dedicated drivable vehicle, a tele-operated vehicle, controlled from a different location.

37. A method for adaptively and selectively tilling soil for agricultural weeding, the method comprising:
obtaining sensory data from a sensory module wherein said sensory module comprises at least one sensor configured and enabled to capture the sensory data of the soil and wherein the sensory data comprises one or more of 2D or 3D images of the soil;
processing and analyzing the sensory data, using a processing module, to generate agricultural data related to the soil;
analyzing the agricultural data and additional data to yield weeding strategy instructions signals;
transmitting the weeding strategy instructions signals to a mechanical module for adaptively and selectively till the soil.

38. The method of claim 37, wherein the mechanical module is towed or shoved by a vehicle

39. The method of claim 37, wherein the sensory data is further analyzed based on vehicle's data and local or external sensors.

40. A mechanical module for tiling or weeding, the mechanical module comprising:
a plurality of implements arranged side by side wherein each implement covers a given width across the width of a mechanical module;
a control module capable of executing a localize adaptive tilling action, using the system of claim 1.
